(19) United States
(12) Patent Application Publication
(10) Pub. No.: US 2012/0121543 A1
(43) Pub. Date: May 17, 2012

(54) ANTI-MICROBIAL AGENT FROM PAENIBACILLUS SP. AND METHODS AND USES THEREOF

(75) Inventors: Ronald Teather, Lethbridge (CA); John Baah, Lethbridge (CA); Karim Naghmouchi, Tunis (TN); James Gibbs Watson, Edmonton (CA); Djamel Drider, Villeneuve d'Ascq Cedex (FR)

(73) Assignee: Best Environmental Technologies Inc., St. Michael (BB)

(21) Appl. No.: 13/296,063

(22) Filed: Nov. 14, 2011

Related U.S. Application Data
(63) Continuation-in-part of application No. PCT/CA09/01808, filed on Dec. 9, 2009.

(51) Int. Cl. A61K 35/74; C07K 2/00; C07K 7/06; C07K 7/64; A23L 3/00; A01N 63/00; A01N 37/18; C07H 21/00; A61P 31/04; A01P 1/00; C12N 1/21; A61K 38/02

(52) U.S. Cl. 424/93.2; 435/252.3; 530/300; 530/328; 530/317; 514/2.4; 514/2.8; 536/23.1; 426/61

(57) ABSTRACT

The present invention provides, in part, a Paenibacillus sp. isolate, designated Paenibacillus polymyxa JB05-01-1, as well as an anti-microbial agent obtained from the bacterium or cell culture supernatant thereof. Compositions, methods and uses are also provided.

### CROSS-REFERENCE TO RELATED APPLICATIONS

The present application is a continuation-in-part of PCT/CA2009/001808, filed on Dec. 9, 2009, and published as WO 2011/069227, the contents of which are incorporated by reference herein in their entirety.

### SEQUENCE LISTING

The following application contains a sequence listing in computer readable format (CRF), submitted as a text file in ASCII format entitled “Sequence_Listing,” created on Nov. 14, 2011, as 3 KB. The content of the CRF is hereby incorporated by reference.

### FIELD OF INVENTION

The invention is in the field of anti-microbial agents. More specifically, the invention relates to anti-microbial agents derived from Paenibacillus.

### BACKGROUND OF THE INVENTION

In response to the increasing prevalence of antibiotic resistance in pathogenic bacteria, the pharmacokinetic properties and safety profiles of many novel antimicrobial peptides have been investigated. Bacteriocins are natural proteinaceous antimicrobial compounds produced by bacteria and active against taxonomically related bacteria (Klaenhammer, 1993). Species that produce bacteriocins have been studied extensively in the hope of finding safe and efficient means of inhibiting the growth of pathogenic bacteria, especially in foods (Cleveland et al. 2001). Bacteriocins produced by Gram-positive staining bacteria, such as lactic acid bacteria, have become a focus of interest as alternatives to conventional antibiotics (Nes et al. 1996). Nisin, the first bacteriocin ever isolated and now widely used as a food additive, was approved by the World Health Organization for use as a food preservative in 1973. This peptide is generally inactive against Gram-negative staining bacteria, imposing a limitation on its effectiveness when major food-borne pathogens such as Escherichia coli, Salmonella and Yersinia are involved (Du and Shen 1999; Zheng et al. 1999). Davies et al. (1998) reported that nisin produced by Lactococcus lactis was thermostable and remained active after treatment at 121° C. for 15 min at pH 3. Nisin is about 4.4 kDa and is stabilized by disulfide bonds.

Polymyxins, a class of antimicrobial agents, are synthesized by a non-ribosomal process. The peptide-synthase-directed condensation reactions by which polymyxins are formed in the cell cytoplasm have been reviewed (Marahiel et al. 1997) and their biosynthesis in a cell-free enzyme system reported (Komura et al. 1985).

Many species within the genus Paenibacillus produce variants of polymyxins, which are generally composed of a cyclic decapeptide with a terminal fatty acid moiety (Martin et al. 2003). Five chemically distinct compounds, polymyxins A to E, differing in amino acid and fatty acid composition have been identified to date. Martin et al. (2003) reported that mattacin activity (800 AU ml⁻¹) produced by P. kobensis M was maximal at 12 h of fermentation. Martin et al. (2003) also reported that mattacin and polymyxin B inhibited all Gram-negative staining species tested including E. coli O157:H7, Salmonella enterica serovar Rubislaw and Vibrio parahemeolyticus G1-166 but both failed to inhibit strains of Listeria and Bacillus.

DeCrescenzo et al. (2007) isolated a new Paenibacillus species (P. amylolyticus C27) that produces polymyxins E1 and E2 (colistin A and B). The new antimicrobial peptides were reported to be effective against Gram-negative staining bacteria such as E. coli, Pseudomonas, Salmonella, and Shigella. DeCrescenzo et al. (2007) also reported that polymyxin E produced by P. amylolyticus C27 inhibited Gram-positive staining bacteria such as Staphylococus aureus ATCC 6538, Enterococcus faecalis ATCC 19433 and Streptococcus pyogenes ATCC 19165.

Zengguo et al. (2007) reported the co-production of polymyxin and lantibiotic by natural isolates of P. polymyxa. The two antimicrobial peptides were reported to display potent activity against many Gram-negative staining bacteria, including E. coli, Pseudomonas aeruginosa and Acinetobacter baumannii, and against Gram-positive food-borne pathogenic bacteria. Zengguo et al. (2007) also reported that polymyxin produced by P. polymyxa OSY-DF is stable from pH 2.0 to 9.0 and retained its activity after a short autoclaving.

Svetoch et al. (2005) reported the isolation of a new class IIa bacteriocin from P. polymyxa NRRL-B-30509, which has been used for the control of Campylobacter in poultry.

Among antimicrobial substances produced by Bacillus polymyxa are polymyxins, which are cyclic peptides with a long hydrophobic tail. Colistin is a polymyxin antibiotic discovered in the late 1940s for the treatment of Gram-negative infections. After several years of clinical use, colistin was associated with significant nephrotoxicity and neurotoxicty (Lim et al. 2010), rendering its use questionable. Colistin has a bactericidal effect against Gram-negative bacteria and acts as a detergent-like molecule (Landman et al. 2008). Recently, its application has returned as the last resort against multidrug-resistant organisms including Pseudomonas aeruginosa, Acinetobacter baumannii, and Klebsiella pneumoniae (Falagas and Kasiakou 2005).

The need for antibiotics with activity against these multidrug-resistant Gram-negative pathogens is urgent and in the absence of viable alternatives, clinicians now recommend colistin treatment when confronted with some multidrug resistant bacterial infections (Lim et al. 2010). This has led to the development of less toxic colistin molecules (Jian Li et al. 2004; Falagas et al., 2006). Colistin formulations available for clinical uses are colistin sulfate (for topical use) and colistin methane sulfonate for parenteral and aerosol therapy (Jian Li et al. 2006).

The coproduction of polymyxin E1 and a 1 antibiotic has been reported for P. polymyxa OSY-DF; a strain isolated from food (He et al. 2007). Polymyxin E1 was active against Gram-negative bacteria, whilst paenibacillin, a proteinaceous compound, exhibited activity against Gram-positive bacteria (He et al. 2007 & 2008). P. kobensis M isolated from soil produced mattacin (polymixin M) (Nathaniel et al. 2003) and Bacillus sp. strain B-60 produced various inhibitory molecules named sattabacin, hydroxysattabacin, sattazolin and methylsattazolin, with antiviral activity against herpes simplex viruses types 1 and 2 (HSV1 and HSV2) (Lampis et al. 1995). Strains of P. polymyxa have been isolated from different ecological niches including food matrices such as butternut squash, potatoes, rice, and wheat flour (Fangio et al. 2010).

### SUMMARY OF THE INVENTION

The present invention provides, in part, an isolated Paenibacillus sp. bacterium comprising SEQ ID NO: 1.

In alternative embodiments, the invention provides an isolated Paenibacillus polymyxa (Strain JB05-01-1) bacterium deposited at the ATCC®) under the terms of the Budapest Treaty and designated Accession Number PTA-10436, or a strain comprising the identifying characteristics thereof. The bacterium may be isolated from a direct-fed microbial product, for example, RE3™.

The bacterium may include an anti-microbial activity, such as an anti-bacterial activity. The anti-bacterial activity may include inhibiting the growth of a Gram-negative staining bacterium, such as one or more of Escherichia sp. (e.g., Escherichia coli such as Escherichia coli RR1, Escherichia coli TB1, or Escherichia coli O157:H7), Pantoea sp. (e.g., Pantoea agglomerans BC1), Pseudomonas sp. (e.g., Pseudomonas fluorescens R73), Butyrivibrio sp. (e.g., Butyrivibrio fibrisolvens OR85), Fibrobacter sp. (e.g., Fibrobacter succinogenes), Salmonella sp. (e.g., Salmonella enteritidis or Salmonella typhi), Shigella sp. (e.g., Shigella dysenteriae), Helicobacter sp. (e.g., Helicobacter pylori), or Campylobacter sp (e.g., Campylobacter jejuni).

The anti-bacterial activity may include inhibiting the growth of a Gram-negative staining bacterium, such as one or more of Escherichia coli RR1, Escherichia coli TB1, and Escherichia coli O157:H7, Pantoea agglomerans BC1, Pseudomonas fluorescens R73, Butyrivibrio fibrisolvens OR85, Fibrobacter succinogenes and Pseudomonas aeruginosa.

In alternative embodiments, the bacterium may not inhibit the growth of a Gram-positive staining bacterium, such as one or more of a Listeria innocua, Listeria, Monocytogenes, Pediococcus acidilactici, Paenibacillus polyrnyxa, Paenibacillus macerans, Bacillus lecheniformis, Bacillus subtilis, Bacillus circulans 9E2, Streptococcus bovis, Enterococcus rnundtii, Staphylococcus aureus, or Lactococcus lactis.

In alternative embodiments, the anti-microbial activity may be sensitive to an enzyme selected from the group consisting of one or more of proteinase K, trypsin, chymotrypsin or lipase; or may be sensitive to sodium dodecyl sulphate (SDS) or urea; or may be sensitive to a temperature in excess of about 90° C. for about 30 minutes; or may be sensitive to a temperature of about 100° C. for about 10 minutes; or may be insensitive to a temperature up to about 80° C. for about 30 minutes; or may be sensitive to acetonitrile and hexane; or may be insensitive to an organic solvent selected from the group consisting of chloroform, propanol, methanol, ethanol and toluene; or may be insensitive to pH, for example, pH ranging from about 2 to about 9.

In alternative embodiments, the invention provides a cell culture including a bacterium as described herein. The cell culture may be a starter culture.

In alternative embodiments, the invention provides a cell culture supernatant derived from growing a bacterium as described herein in a cell culture medium. The supernatant may include an anti-microbial activity, such as an anti-bacterial activity.

In alternative embodiments, the invention provides an anti-microbial agent isolated from a bacterium, cell culture, or cell culture supernatant as described herein.

The anti-microbial agent may include a peptide, such as a lipopeptide. The anti-microbial agent may include a molecular weight between about 1000 daltons to about 2500 daltons. The anti-microbial agent may be a polymyxin. The anti-microbial agent may be colistin A and/or colistin B.

In alternative embodiments, the invention provides a bacterium, cell culture, cell culture supernatant, or anti-microbial agent as described herein.

In alternative embodiments, the invention provides a pharmaceutical, veterinary, cosmetic or hygiene composition including a bacterium, cell culture, cell culture supernatant, or anti-microbial agent as described herein and a suitable carrier.

In alternative embodiments, the invention provides a food or feed additive comprising a bacterium, cell culture, cell culture supernatant, or anti-microbial agent as described herein.

In alternative embodiments, the invention provides a packaging material comprising a bacterium, cell culture, cell culture supernatant, or anti-microbial agent as described herein.

In alternative embodiments, the invention provides a kit comprising a bacterium, cell culture, cell culture supernatant, or anti-microbial agent as described herein together with instructions for use in inhibiting growth of a micro-organism.

In alternative embodiments, the invention provides a method of producing an anti-microbial agent, by providing a live Paenibacillus sp. bacterium comprising SEQ ID NO: 1; and culturing the live Paenibacillus sp. bacterium in a cell culture medium, under conditions suitable for production of the anti-microbial agent.

In alternative embodiments, the invention provides a method of producing an anti-microbial agent, by providing a live Paenibacillus polymyxa (Strain JB05-01-1) bacterium or a strain comprising the identifying characteristics thereof; and culturing the live Paenibacillus polymyxa (Strain JB05-01-1) bacterium or a strain comprising the identifying characteristics thereof in a cell culture medium, under conditions suitable for production of the anti-microbial agent.

The methods may further include isolating the anti-microbial agent from the bacterium. The culturing may be performed under conditions suitable for secretion of the anti-microbial agent into the cell culture medium. The methods may further include separating the bacterium from the cell culture medium to provide a cell culture supernatant comprising the anti-microbial agent. The methods may further include isolating the anti-microbial agent from the cell culture supernatant.

In alternative embodiments, the invention provides an anti-microbial agent produced by the methods as described herein. The anti-microbial agent may include a peptide, such as a lipopeptide. The anti-microbial agent may include a molecular weight between about 1000 daltons to about 2500 daltons. The anti-microbial agent may be a polymyxin. The anti-microbial agent may be colistin A and/or colistin B.

The anti-microbial agent may include an anti-microbial activity selected from one or more of sensitivity to proteinase K, trypsin, chymotrypsin and lipase, sodium dodecyl sulphate (SDS), urea, acetonitrile, or hexane; insensitivity to chloroform, propanol, methanol, ethanol or toluene; insensitivity to pH; sensitivity to a temperature in excess of about 90° C. for about 30 minutes; sensitivity to a temperature of about 100° C. for about 10 minutes; insensitivity to a temperature upto about 80° C. for about 30 minutes; inhibition of growth of a Gram-negative staining bacterium; or no inhibition of growth of a Gram-positive staining bacterium.

In alternative embodiments, the invention provides a method of inhibiting the growth of a microorganism in a subject or substance in need thereof by administering or applying an effective amount of the bacterium, cell culture, cell culture supernatant or anti-microbial agent, as described herein, to the subject or substance. The inhibition of growth may be selective.

The microorganism may be a bacterium, such as a Gram-negative staining bacterium, such as one or more of one or more of Escherichia sp. (e.g., Escherichia coli such as Escherichia coli RR1, Escherichia coli TB1, or Escherichia coli O157:H7), Pantoea sp. (e.g., Pantoea agglomerans BC1), Pseudomonas sp. (e.g., Pseudomonas fluorescens R73 or Pseudomonas aeruginosa), Butyrivibrio sp. (e.g., Butyrivibrio fibrisolvens OR85), Fibrobacter sp. (e.g., Fibrobacter succinogenes), Salmonella sp. (e.g., Salmonella enteritidis or Salmonella typhi), Shigella sp. (e.g., Shigella dysenteriae), Helicobacter sp. (e.g., Helicobacter pylori), or Campylobacter sp (e.g., Campylobacter jejuni).

The Gram-negative staining bacterium, may be one or more of Escherichia coli RR1, Escherichia coli TB1, and Escherichia coli O157:H7, Pantoea agglomerans BC 1, Pseudomonas fluorescens R73, Butyrivibrio fibrisolvens OR85, Fibrobacter succinogenes and Pseudomonas aeruginosa.

The bacterium may be a pathogenic bacterium, such as a food-borne pathogenic bacterium. The microorganism may be a food-borne pathogenic micro-organism or a food-spoilage micro-organism.

The subject may be an animal, such as a human or an agricultural animal (e.g., cow, horse, pig, sheep, goat, chicken, turkey, duck, goose, fish, or crustacean).

The substance may be a cosmetic, hygiene, feed or food product (e.g., dairy or meat product) or packaging material thereof.

In alternative embodiments, the invention provides an isolated nucleic acid molecule including SEQ ID NO: 1.

In alternative embodiments, the invention provides the use of an effective amount of a bacterium, cell culture, cell culture supernatant or anti-microbial agent, as described herein for inhibiting the growth of a microorganism in a subject or substance in need thereof.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a graph showing Kinetics of P. polymyxa JB05-01-1 growth and production of “JB05-01-1” during stirred batch culture in Luria-Bertani broth at 30° C. Filled diamond is culture optical density; open triangle is inhibitory activity expressed as AU/ml, determined by micro-dilution assay

FIG. 2 is a graph showing the antimicrobial activity against E. coli RR1. DiVerent amounts of crude P. polymyxa culture supernatant (1 mg/ml) containing antimicrobial substance(s) “JB05-01-1” at 0 (open diamond), 32 (open square), and 96 (open triangle) AU/ml or polymyxin B (0.1 μg/ml) (open circle) were added to exponentially growing culture of Escherichia coli RR1 in tryptic soy broth at 30° C.

FIG. 2A is optical density at 600 nm; FIG. 2B is viable cell counts (CFU, colony forming unit). Arrows show crude antimicrobials peptides addition

FIG. 3 is a photograph showing SDS-PAGE profiles of partially purified antimicrobial substance produced by Paenibacillus polymyxa JB05-01-1. FIG. 3A gel was stained with Coomassie Brilliant Blue staining R-250. FIG. 3B gel recovered with TSBA pre-overlaid with E. coli RR1. Lane 1 contains the molecular weight markers (in KD), Lane 2 contains partly purified antimicrobial substance JB05-01-1. Arrows in gel FIG. 3A and FIG. 3B show a band and a zone of inhibition respectively.

FIG. 4 is a partial sequence of P. polymyxa JB05-01-1 16S rRNA gene (GenBank Accession Number GQ184435; SEQ ID NO: 1).

FIG. 5 is a Fast Protein Liquid Chromatography (FPLC) of purified antimicrobials peptides produced by P. polymyxa JB05-01-1 eluted at 30% acetonitrile (CH₃CN), 1% TFA.

FIG. 6 is a mass spectrometry analysis of purified Colistin A and B produced by P. polymyxa JBO5-01-1, and structure determination. Molecular weights were determined using MS analysis (Colistin A: 1169Da: Colistin B: 1155 Da). MS/MS spectrums relevant to the species at m/z 585.3 and 578.4 observed in the chromatograms of FIG. 6A and FIG. 6B. The assignments of the different peaks are shown for the corresponding peptide sequence. FIG. 6C is a chemical structure of fatty acid (FA): Colistin A, 6-methyloctanoyl; Colistin B, 6-methylhepatanoyl.

### DETAILED DESCRIPTION

The present invention provides, in part, a Paenibacillus sp. isolate, designated Paenibacillus polymyxa JB05-01-1 (deposited on Oct. 21, 2009, with the American Type Culture Collection (ATCCC) under the terms of the Budapest Treaty and assigned Accession Number PTA-10436) obtained from an animal feed additive and identified by amplification and sequencing of the 16S rRNA gene. Characterization of the physical properties and anti-microbial activities of a substance secreted into the culture supernatant of a Paenibacillus polymyxa JB05-01-1 cell culture resulted in the identification of at least one anti-microbial agent.

Anti-Microbial Agents

An “anti-microbial agent,” as used herein, refers to an agent that exhibits one or more “anti-microbial activity” i.e., any activity that inhibits the growth of a micro-organism. By “inhibit,” “inhibition” or “inhibiting” is meant to destroy, prevent, control, decrease, slow or otherwise interfere with the growth or survival of a micro-organism by at least about 10% to at least about 100%, or any value therebetween for example about 10%, 15%, 20%, 25%, 30%, 35%, 40%, 45%, 50%, 55%, 60%, 65%, 70%, 75%, 80%, 85%, 90%, 95%, 99%, or 100% when compared to the growth or survival of the micro-organism in the absence of the anti-microbial agent. In alternative embodiments, by “inhibit” “inhibition” or “inhibiting” is meant to destroy, prevent, control, decrease, slow or otherwise interfere with the growth or survival of a micro-organism by at least about 1-fold or more, for example, about 1.5-fold to about 100-fold, or any value therebetween for example about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when compared to the growth or survival of the micro-organism in the absence of the anti-microbial agent. In alternative embodiments, the “inhibition” may be more than 100-fold. In alternative embodiments, the “inhibition” may be substantially complete inhibition of growth i.e., the growth rate may be reduced to about zero in the presence of the anti-microbial agent, and the anti-microbial agent may cause death of a micro-organism, when compared to the growth or survival of the micro-organism in the absence of the anti-microbial agent. Accordingly, an anti-microbial agent may be microbicidal or may be microbistatic.

In some embodiments, the anti-microbial agent may be an anti-bacterial agent i.e., an agent that exhibits one or more “anti-bacterial activity” i.e., any activity that inhibits the growth of a bacterium. In alternative embodiments, the anti-bacterial agent may be bactericidal or bacteriostatic.

In some embodiments, an anti-bacterial agent according to the invention may selectively inhibit the growth of a Gram-negative staining bacterium.

By “selectively inhibit” “selective inhibition” or “selectively inhibiting” is meant to destroy, prevent, control, decrease, slow or otherwise interfere with the growth or survival of a Gram-negative staining bacterium by at least about 10% to at least about 100%, or any value therebetween for example about 10%, 15%, 20%, 25%, 30%, 35%, 40%, 45%, 50%, 55%, 60%, 65%, 70%, 75%, 80%, 85%, 90%, 95%, 99%, or 100% when compared to the growth or survival of a Gram-positive staining bacterium. In alternative embodiments, by “selectively inhibit” “selective inhibition” or “selectively inhibiting” is meant to destroy, prevent, control, decrease, slow or otherwise interfere with the growth or survival of a Gram-negative staining bacterium by at least about 1-fold or more, for example, about 1.5-fold to about 100-fold, or any value therebetween for example about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when compared to the growth or survival of a Gram-positive staining bacterium. In alternative embodiments, the “selective inhibition” may be more than 100-fold. In alternative embodiments, the “selective inhibition” may be substantially complete inhibition of growth of a Gram-negative staining bacterium i.e., the growth rate could be reduced to about zero and the anti-bacterial agent may cause death of a Gram-negative staining bacterium when compared to the growth or survival of Gram-positive staining bacterium.

Gram-negative staining bacteria include without limitation Escherichia sp., Pantoea sp., Pseudomonas sp., Salmonella sp., Shigella sp., Pseudomonas sp., Helicobacter sp., Butyrivibrio sp., Fibrobacter sp. or Campylobacter sp. Examples of Gram-negative staining bacteria species include without limitation Escherichia coli (e.g., Escherichia coli RR1, Escherichia coli TB1, Escherichia coli O157:H7), Pantoea agglomerans, Pseudomonas fluorescens, Pseudomonas aeruginosa, Salmonella enteritidis, Salmonella typhi, Shigella dysenteriae, Helicobacter pylori, Butyrivibrio fibrisolvens, Fibrobacter succinogenes or Campylobacter jejuni.

In alternative embodiments, an anti-microbial agent according to the invention does not substantially inhibit the growth of Gram-positive staining bacteria, such as one or more of Listeria innocua, Listeria, Monocytogenes, Pediococcus acidilactici, Paenibacillus polymyxa, Paenibacillus macerans, Bacillus lecheniformis, Bacillus subtilis, Bacillus circulans 9E2, Streptococcus bovis, Enterococcus mundtii, Staphylococcus aureus, or Lactococcus lactis.

In some embodiments, an anti-microbial agent according to the invention may be sensitive or insensitive to various treatments. By “sensitive” or “sensitivity” is meant loss or reduction of anti-microbial activity by at least about 10% to at least about 100%, or any value therebetween for example about 10%, 15%, 20%, 25%, 30%, 35%, 40%, 45%, 50%, 55%, 60%, 65%, 70%, 75%, 80%, 85%, 90%, 95%, 99%, or 100% when an anti-microbial agent is subjected to a particular treatment, when compared to anti-microbial activity in the absence of the treatment. In alternative embodiments, by “sensitive” or “sensitivity” is meant loss or reduction of anti-microbial activity by at least about 1-fold or more, for example, about 1.5-fold to about 100-fold, or any value therebetween for example about 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95-fold when an anti-microbial agent is subjected to a particular treatment, when compared to anti-microbial activity in the absence of the treatment. In alternative embodiments, the “sensitivity” may include loss or reduction of anti-microbial activity of more than 100-fold.

It is to be understood that sensitivity may vary with the time of treatment. In alternative embodiments, the time of treatment may range from a few minutes to many hours. For example, the time of treatment may be about 5 minutes to over 25 hours, such as 5, 10, 15, 20, 25, 30, 35, 40, 45, 50, or 55 minutes or any value therebetween, or such as 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0 hours, or any value therebetween. Anti-microbial activity may be tested by standard methods such as agar diffusion tests and micro-dilution assay as described herein, or by other standard methods such as disk diffusion, agar dilution, or through the use of automated instrumental testing systems (see, for example. Manual of Clinical Microbiology. 1995. P. M. Murray (ed). ASM Press, Washington, D.C.).

By “insensitive” or “insensitivity” is meant no substantial observable effect or sensitivity when an anti-microbial agent is subjected to a particular treatment, when compared to anti-microbial activity in the absence of the treatment. In alternative embodiments, by “insensitive” or “insensitivity” is meant an observable effect of less than about 10%, for example about 0%, 1%, 2%, 3%, 4%, 5%, 6%, 7%, 8%, or 9% when an anti-microbial agent is subjected to a particular treatment, when compared to anti-microbial activity in the absence of the treatment.

In some embodiments, an anti-microbial agent according to the invention may be sensitive to treatment with proteases such as proteinase K, trypsin, chymotrypsin or with a lipase.

In some embodiments, an anti-microbial agent according to the invention may be sensitive to treatment with surfactants such as sodium dodecyl sulphate (SDS), chaotropics agents such as urea, or solvents such as acetonitrile or hexane.

In some embodiments, an anti-microbial agent according to the invention may be insensitive to an organic solvent such as chloroform, propanol, methanol, ethanol or toluene.

In some embodiments, an anti-microbial agent according to the invention may be insensitive to pH, for example, pH ranging from about 2 to about 9.

In some embodiments, an anti-microbial agent according to the invention may be sensitive to a temperature in excess of about 80° C. In some embodiments, an anti-microbial agent according to the invention may be sensitive to a temperature in excess of about 90° C. Accordingly, in some embodiments, an anti-microbial agent according to the invention may be sensitive to a temperature in excess of about 90° C. when exposed for at least about 30 minutes. In alternative embodiments, an anti-microbial agent according to the invention may be sensitive to a temperature of about 100° C. when exposed for at least about 10 minutes.

In some embodiments, an anti-microbial agent according to the invention may be insensitive to a temperature upto about 80° C. when exposed for about 30 minutes.

In particular embodiments, sensitivity of an anti-microbial agent according to the invention may include: the loss (about 100%) of anti-microbial activity after treatment of a composition including the anti-microbial agent with proteinase K for 10 minutes at 100° C.; the reduction of anti-microbial activity to about 83% or about 75% by trypsin and chymotrypsin, respectively, or to about 62% by lipase; the reduction of anti-microbial activity to about 66% after SDS treatment and about 58% after Urea treatment.

In some embodiments, the molecular weight of an anti-microbial agent according to the invention may be about 1,000 Da to about 2,500 Da. In some embodiments, an anti-microbial agent according to the invention may include more than one molecule having a molecular weight in the range of about 1,000 Da to about 2,500 Da. The agent may be a peptide, for example, a lipopeptide.

In some embodiments, an anti-microbial agent according to the invention may be a peptidic compound including for example a nonproteinaceous amino acid, such as a D-amino acid or a hydroxy acid and/or may be modified for example by N methylation, acylation, glycosylation, or heterocyclic ring formation.

In some embodiments, an anti-microbial agent according to the invention may be a polymyxin. By “polymyxin” is meant a peptide having anti-microbial activity. In general, the structure of a polymyxin may include a cyclic peptide e.g., a cyclic decapeptide, with a terminal fatty acid moiety, that is capable of inhibiting the growth of a micro-organism such as a Gram-negative staining bacterium.

In some embodiments, an anti-microbial agent according to the invention may be one or both of colistin A and colistin B. By “colistin A/B” is meant a polymyxin having anti-microbial activity. In general, colistin A has a molecular weight of about 1169 Da and includes fatty acid moiety 6-methyloctanoyl. Colistin B generally has a molecular weight of 1155 Da and includes fatty acid moiety 6-methylhepatanoyl.

An anti-microbial agent according to the invention may include an anti-microbial agent produced by Paenibacillus polymyxa JB05-01-1, ATCC® Accession Number PTA-10436, or by a naturally-occurring bacterium that includes the 16S rRNA sequence of SEQ ID NO:1.

In some embodiments, an anti-microbial agent may include one or more compounds.

An anti-microbial agent may be present in a cell, or crude extract, cell culture, or cell culture supernatant thereof. The cell may be a Paenibacillus polymyxa JB05-01-1 cell or a naturally-occurring bacterium that includes the 16S rRNA sequence of SEQ ID NO:1.

### Methods of Obtaining and Producing Anti-Microbial Agents

Anti-microbial agent(s) may be obtained from Paenibacillus polymyxa JB05-01-1 or from other sources. For example, RE3 (Basic Environmental Systems & Technology Inc. Edmonton, AB, Canada) is a direct-fed microbial product used to improve in vitro ruminal fermentation of barley grain/barley silage-based diets and includes a non-sterile liquid formulation containing L. paracasei and L. lactis cultures and their fermentation products. Paenibacillus polymyxa JB05-01-1 was obtained by culturing a sample of RE3™. Other anti-microbial agents may similarly be found by routine screening for isolates that include the 16S rRNA sequence of SEQ ID NO:1 as described herein or known in the art.

Anti-microbial agent(s) may be produced by growing or culturing Paenibacillus polymyxa JB05-01-1 or a bacterium that includes the 16S rRNA sequence of SEQ ID NO:1 in an appropriate cell culture medium under conditions suitable for production of anti-microbial agent(s) as described herein or known in the art. In alternative embodiments, Paenibacillus polymyxa JB05-01-1 or a bacterium that includes the 16S rRNA sequence of SEQ ID NO:1 may be grown in an appropriate cell culture medium under conditions suitable for secretion of anti-microbial agent(s) into the cell culture supernatant as described herein or known in the art.

The cell culture medium may be a minimal medium or a complete medium. In some embodiments, the cell culture medium may be LB medium (Luria-Bertani medium). The medium may be a liquid medium or may be a solid or semi-solid medium, such as nutrient broth or agar, or tryptic soy broth or agar. In general, the cell culture medium includes a carbon/energy source, NH₄—N, and biotin.

The cell culture conditions (e.g., temperature, time, etc.) may be varied as appropriate to optimize growth and/or production of the anti-microbial agent(s).

In some embodiments, the temperature may range from about 5° C. to about 40° C., such as 10° C., 15° C., 20° C., 25° C., 30° C., or 35° C., or any value therebetween. In alternative embodiments, the temperature may be about 30° C.

In some embodiments, the time may range from about 5 hours to about 48 hours or any value therebetween. In alternative embodiments, the time may be greater than 48 hours. In alternative embodiments, the time may be about 20 hours.

The cell culture conditions may be aerobic or anaerobic.

Standard separation processes may be used to obtain a substantially pure preparation of an anti-microbial agent. An agent or compound is “substantially pure” or “isolated” when it is separated from the components that naturally accompany it.

Typically, an anti-microbial agent or compound is substantially pure when it is at least 10%, 20%, 30%, 40%, 50%, or 60%, more generally 70%, 75%, 80%, or 85%, or over 90%, 95%, or 99% by weight, of the total material in a sample. Thus, for example, a substantially pure preparation or culture of a cell expressing an anti microbial agent, such as a Paenibacillus polymyxa JB05-01-1 cell or a naturally-occurring bacterium that includes the 16S rRNA sequence of SEQ ID NO:1, is a preparation of cells or “cell culture” in which contaminating cells that are not a Paenibacillus polymyxa JB05-01-1 cell, or do not have the desired 16S rRNA sequence of SEQ ID NO:1, or do not express an anti-microbial agent as described herein, constitute less than 1%, 5%, 10%, 20%, 30%, 40%, or 50%, of the total number of cells in the preparation. In some embodiments, a substantially pure Paenibacillus polymyxa JB05-01-1 cell or a substantially pure naturally-occurring bacterium that includes the 16S rRNA sequence of SEQ ID NO:1, is a preparation of cells or “cell culture” that contains 100% of such cells.

In some embodiments, an anti-microbial agent that is isolated by known purification techniques, or isolated as described herein, will be generally be substantially free from its naturally associated components. A substantially pure anti-microbial agent can be obtained, for example, by extraction from a natural source such as a Paenibacillus polymyxa JB05-01-1 cell or a naturally-occurring bacterium that includes the 16S rRNA sequence of SEQ ID NO:1.

In some instances, an anti-microbial agent according to the invention will form part of a composition, for example, a crude extract containing other substances. For example, an anti-microbial agent may be present in a crude extract of a Paenibacillus polymyxa JB05-01-1 cell or a naturally-occurring bacterium that includes the 16S rRNA sequence of SEQ ID NO:1 that may also contain the other naturally occurring components found in such a cell. A crude extract of a Paenibacillus polymyxa JB05-01-1 cell or a naturally-occurring bacterium that includes the 16S rRNA sequence of SEQ ID NO:1 may be prepared by routine procedures, for example, disruption of the cells using standard mechanical or non-mechanical techniques such as freeze-thaw techniques, osmotic shock, enzyme (e.g., lysozyme) treatment, ultrasonication, liquid extrusion, etc., which may be followed by removal of the cell debris by for example centrifugation.

In alternative embodiments, an anti-microbial agent may be present in a cell culture supernatant, such as a supernatant obtained from growing a Paenibacillus polymyxa JB05-01-1 cell or a naturally-occurring bacterium that includes the 16S rRNA sequence of SEQ ID NO:1 in a suitable cell culture medium under conditions suitable for secretion of the anti-microbial agent into the supernatant. The term “culture supernatant” refers to the liquid broth remaining when cells grown in a medium are separated from the culture medium by for example centrifugation, filtration, sedimentation, or other means well known in the art. As an example, if the anti-microbial agent(s) is to be isolated from cell culture supernatant, a salt such as ammonium sulphate may be used at various concentrations, initially. Residual ammonium sulphate may then be removed by dialysis against water. The suspended precipitate containing one or more than one antimicrobial compound may be chromatographed on a column such as an ion exchanger, and the various compounds in the culture supernatant may be separated by monitoring absorbance at 280 nm. Active fractions can be determined from among the compounds thus separated, and selected on the basis of the efficacy with which aliquots thereof kill or inhibit the growth of microbes such as bacterial cells, i.e. the indicator strain, known to be sensitive to the anti-microbial agent(s). Active fractions may then be pooled. Further purification may be carried out by high performance liquid chromatography (HPLC) based on the charge of the compound. The various peaks obtained by monitoring absorbance at 280 nm may be separated and again tested for activity against the indicator strain. Purity can be measured using any appropriate method such as column chromatography, gel electrophoresis, HPLC, etc.

The anti-microbial agent may be isolated from the cell culture supernatant using solid phase extraction with columns such as Amberlite XAD-16 column and Sep-Pak C18 Column. The active fraction eluted from the column(s) may be further purified based on molecular weight separation using a Fast Protein Liquid Chromatography (FPLC) system.

A person skilled in the art would understand that other conventional concentration, purification or fractionation methods may be used to obtain one or more isolated or substantially purified anti-microbial agent(s) or partially purified fractions exhibiting an anti-microbial activity from whole or lysed cells or from cell culture supernatant. Typical methods include, without limitation, size exclusion or ion exchange chromatography, ammonium sulfate, alcohol, or chloroform extraction, or centrifugation with size filters.

The anti-microbial activity of an anti-microbial agent may be determined by routine methods or as described herein. For example, anti-microbial activity may be detected by agar diffusion tests or micro-dilution assay.

### Pharmaceutical, Veterinary, Nutritional, Cosmetic and Other Uses

Anti-microbial agent(s) according to the invention may be used in a variety of applications in which inhibition of growth of a micro-organism, such as a bacterium, is desirable. Such applications include, without limitation, pharmaceutical and veterinary applications (e.g., for the treatment of a microbial infection), nutritional supplements and animal feed, personal care (cosmetic or hygiene) applications, etc. In alternative embodiments, anti-microbial agent(s) according to the invention may be used to inhibit the growth of a microorganism (e.g., a bacterium) involved in the spoilage of food or other products.

Food spoilage micro-organisms include without limitation one or more species of Clostridium, Pseudomonas, Porteus, Chromobacterium, Chromobacterium, Lactobacillus, Penicillium, Aspergillus, Rhizopus, Micrococcus, Bacillus, Streptococcus, Pediococcus, Leuconostoc, Chromobacterium, Halobacterium, Alcaigenes, Xanthomonas, Botryitis, Aerobacter, Cornebaclerium, Arthrobacter, Microbacterium, Serratia, etc.

The micro-organism may be a pathogenic micro-organism. The bacterium may a pathogenic bacterium, such as food-borne pathogenic bacterium. Food-borne pathogenic bacteria include without limitation one or more species of Staphylococcus, Escherichia, Listeria, Monocytogenes, Salmonella, Streptococcus, Vibrio, Campylobacter, Enterobacter, Shigella, etc.

The bacterium may include a Gram-negative staining bacterium. Gram-negative staining bacteria include without limitation one or more species of Escherichia sp., Pantoea sp., Pseudomonas sp., Salmonella sp., Shigella sp., Helicobacter sp., Campylobacter sp. or Butyrivibrio sp., and Fibrobacter sp. Examples of Gram-negative bacteria species include without limitation Escherichia coli (e.g., Escherichia coli RR1, Escherichia coli TB1, Escherichia coli O157:H7), Pantoea agglomerans, Pseudomonasfluorescens, Pseudomonas aeruginosa, Salmonella enteritidis, Salmonella typhi, Shigella dysenteriae, Helicobacter pylori, Campylobacter jejuni, Butyrivibrio fibrisolvens, or Fibrobacter succinogenes.

Other examples of bacteria include without limitation gram-negative rods such as enteric Gram-negative staining rods, curved. Gram-negative staining rods, parvobacteria and Haemophilus, Gram-negative staining cocci such as Neisseria, non-sporing anaerobes, and bacteria such as spirochaetes, rickettsia and chlamydia.

Examples of microial infections, such as bacterial infections, include without limitation chlamydia, gonorrhea, salmonellosis, shigellosis, tuberculosis, syphilis, bacterial pneumonia, bacterial sepsis (bacteremia), bacterial urinary tract infections, vaginosis, bacterial upper respiratory tract infections, bacterial meningitis, bacterial enteritis, diphtheria, legionellosis, pertussis, scarlet fever, toxic shock syndrome, psittacosis, otitis media, lyme disease, etc.

### Pharmaceutical, Veterinary, Nutritional and Other Compositions, Dosages, and Administration

Anti-microbial agents of the invention can be provided alone or in combination with other compounds, in the presence of any pharmaceutically, veterinarily or cosmetically acceptable carrier, diluent, and/or excipient in a form suitable for administration to animals, for example, humans, cattle, sheep, pigs, poultry, etc. if desired, administration or application of an anti-microbial agent according to the invention may be combined with more traditional and existing anti-microbial therapies, treatments, supplements, or additives, or with other desirable therapies, treatments, supplements, or additives.

Anti-microbial agents according to the invention may be provided chronically or intermittently. “Chronic” administration refers to administration of the anti-microbial agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical or veterinary practice may be employed to provide suitable formulations or compositions to administer the anti-microbial agent(s) to subjects suffering from or presymptomatic for a microbial infection. Any appropriate route of administration may be employed, for example, parenteral, intravenous, subcutaneous, intramuscular, intracranial, intraorbital, ophthalmic, intraventricular, intracapsular, intraspinal, intrathecal, intracisternal, intraperitoneal, intranasal, intra-anal, intravaginal, aerosol, topical, or oral administration. Therapeutic formulations may be in the form of liquid solutions, syrups, or suspensions; for oral administration, formulations may be in the form of tablets or capsules; and for intranasal formulations, in the form of powders, nasal drops, or aerosols; and topical formulations may come in the form of balms, creams, and lotions.

Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the anti-microbial agent(s) are administered to a subject in an amount sufficient to inhibit the growth of a micro-organism.

An “effective amount” of an anti-microbial agent(s) according to the invention includes a therapeutically effective amount or a prophylactically effective amount. A “therapeutically effective amount” refers to an amount effective, at dosages and for periods of time necessary, to achieve the desired therapeutic result, such as inhibition of the growth of a micro-organism. A therapeutically effective amount of an anti-microbial agent(s) may vary according to factors such as the disease state, age, sex, and weight of the individual or subject, and the ability of the anti-microbial agent(s) to elicit a desired response in the individual or subject. Dosage regimens may be adjusted to provide the optimum therapeutic or prophylactic response. A therapeutically effective amount is also one in which any toxic or detrimental effects of the anti-microbial agent(s) are outweighed by the therapeutically beneficial effects. A “prophylactically effective amount” refers to an amount effective, at dosages and for periods of time necessary, to achieve the desired prophylactic result, such as such as inhibition of the growth of a micro-organism. Typically, a prophylactic dose is used in subjects prior to or at an earlier stage of disease, so that a prophylactically effective amount may be less than a therapeutically effective amount. An exemplary range for therapeutically or prophylactically effective amounts of an anti-microbial agent(s) may be any value from about 0.1 nM to about 0.1M, for example about 0.1 nM to about 0.05M, about 0.05 nM to about 15 μM or about 0.01 nM-to about.

It is to be noted that dosage values may vary with the severity of the condition to be alleviated. For any particular subject, specific dosage regimens may be adjusted over time according to the individual need and the professional judgement of the person administering or supervising the administration of the anti-microbial agent(s). Dosage ranges set forth herein are exemplary only and do not limit the dosage ranges that may be selected by medical or veterinary practitioners. The amount of active anti-microbial agent(s) in the composition may vary according to factors such as the disease state, age, sex, and weight of the individual. Dosage regimens may be adjusted to provide the optimum therapeutic or prophylactic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. As used herein, a subject may be a mammal, an agricultural (e.g., farm) or domestic animal, an experimental animal or any animal that may benefit from the anti-microbial agents as described herein. For example, a subject may include a human, non-human primate, rat, mouse, cow, horse, pig, sheep, goat, chicken, turkey, duck, goose, dog, cat, fish, crustacean, etc.

In general, anti-microbial agent(s) of the invention should be used without causing substantial toxicity. Toxicity of the anti-microbial agent(s) of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the anti-microbial agent(s).

In alternative embodiments, an “effective amount” of an anti-microbial agent according to the invention includes an amount effective to inhibit the growth of a micro-organism, such as a bacterium. It is to be understood that such amounts need not be therapeutic or prophylactic amounts, as long as the amount of the anti-microbial agent is capable of inhibiting the growth of a micro-organism, such as a bacterium, in the context in which it is administered or applied, for example, for prevention of food spoilage, etc.

In alternative embodiments, an anti-microbial agent according to the invention may be provided in a cell, for example a substantially pure Paenibacillus polymyxa JB05-01-1 cell or a substantially pure naturally-occurring bacterium that includes the 16S rRNA sequence of SEQ ID NO:1, or a cell culture thereof. The cell may be provided in a liquid, or may be frozen or dried, e.g., freeze-dried. The cell culture may be concentrated. The cell culture may be a “starter” culture for example for a dairy product (e.g., milk, cheese, etc.), or for selective media in a laboratory.

The anti-microbial agent may be provided in a therapeutic, veterinary, hygiene, cosmetic, food, drink or feed product. In alternative embodiments, the anti-microbial agent may be provided in the packaging material for, for example, a therapeutic, veterinary, hygiene, cosmetic, food, drink or feed product. The packaging material may include without limitation, plastic, film, styrofoam, etc.

In alternative embodiments, an anti-microbial agent according to the invention may be provided in a kit that may optionally include additional anti-microbial agents or desirable therapies, treatments, supplements, or additives, optionally with instructions for use thereof.

In alternative embodiments, an anti-microbial agent according to the invention may be provided as a nutritional or food additive, or feed supplement or additive.

A “nutritional additive” or “food additive” refers to a substance that is added to food, generally to affect the characteristics of the food, such as spoilage. A food additive may be “direct” in that it is directly added to food for example to inhibit growth of a micro-organism. A food additive may be considered “indirect” when it is exposed to food during processing, packaging, or storage but is not present in the final food product. The term “feed additive” or “feed supplement” refers to products used in animal nutrition for purposes of improving the quality of feed, or to improve the animals' performance and health, e.g. providing enhanced digestibility of the feed materials or inhibiting the growth of micro-organisms. An example of an animal feed additive is a direct-fed microbial product which refers to a mono or mixed culture of live micro-organisms, which when applied to a host affects beneficially the host by improving the properties of the indigenous microflora. A non-limiting example of a direct-fed microbial product is RE3™ from Basic Environmental Systems Technology Inc. Edmonton, AB, Canada. In some embodiments, RE3™ may be specifically excluded from a feed additive according to the invention.

In alternative embodiments, anti-microbial agents of the invention can be provided in combination with other feed or nutritional supplements or additives. For example, at least one supplement or additive, such as listed herein, can be included for consumption with the anti-microbial agent of the invention and may have, for example, antioxidant, dispersant, antimicrobial, or solubilizing properties.

A suitable antioxidant is, for example, vitamin C, vitamin E or rosemary extract. A suitable dispersant is, for example, lecithin, an alkyl polyglycoside, polysorbate 80 or sodium lauryl sulfate. A suitable antimicrobial is, for example, sodium sulfite or sodium benzoate. A suitable solubilizing agent is, for example, a vegetable oil such as sunflower oil, coconut oil, and the like, or mono-, di- or tri-glycerides. Additives include vitamins such as vitamin A (retinol, retinyl palm itate or retinol acetate), vitamin B1 (thiamin, thiamin hydrochloride or thiamin mononitrate), vitamin B2 (riboflavin), vitamin B3 (niacin, nicotinic acid or niacinamide), vitamin B5 (pantothenic acid, calcium pantothenate, d-panthenol or d-calcium pantothenate), vitamin B6 (pyridoxine, pyridoxal, pyridoxamine or pyridoxine hydrochloride), vitamin B12 (cobalamin or cyanocobalamin), folic acid, folate, folacin, vitamin H (biotin), vitamin C (ascorbic acid, sodium ascorbate, calcium ascorbate or ascorbyl palmitate), vitamin D (cholecalciferol, calciferol or ergocalciferol), vitamin E (d-alpha-tocopherol, d-beta-tocopherol, d-gamma-tocopherol, d-delta-tocopherol or d-alpha-tocopheryl acetate) and vitamin K (phylloquinone or phytonadione). Other additives include minerals such as boron (sodium tetraborate decahydrate), calcium (calcium carbonate, calcium caseinate, calcium citrate, calcium gluconate, calcium lactate, calcium phosphate, dibasic calcium phosphate or tribasic calcium phosphate), chromium (GTF chromium from yeast, chromium acetate, chromium chloride, chromium trichloride and chromium picolinate) copper (copper gluconate or copper sulfate), fluorine (fluoride and calcium fluoride), iodine (potassium iodide), iron (ferrous fumarate, ferrous gluconate or ferrous sulfate), magnesium (magnesium carbonate, magnesium gluconate, magnesium hydroxide or magnesium oxide), manganese (manganese gluconate and manganese sulfate), molybdenum (sodium molybdate), phosphorus (dibasic calcium phosphate, sodium phosphate), potassium (potassium aspartate, potassium citrate, potassium chloride or potassium gluconate), selenium (sodium selenite or selenium from yeast), silicon (sodium metasilicate), sodium (sodium chloride), strontium, vanadium (vanadium sulfate) and zinc (zinc acetate, zinc citrate, zinc gluconate or zinc sulfate). Other additives include amino acids, peptides, and related molecules such as alanine, arginine, asparagine, aspartic acid, carnitine, citrulline, cysteine, cystine, dimethylglycine, gamma-aminobutyric acid, glutamic acid, glutamine, glutathione, glycine, histidine, isoleucine, leucine, lysine, methionine, ornithine, phenylalanine, proline, serine, taurine, threonine, tryptophan, tyrosine and valine. Other additives include animal extracts such as cod liver oil, marine lipids, shark cartilage, oyster shell, bee pollen and d-glucosamine sulfate. Other additives include unsaturated free fatty acids such as γ-linoleic, arachidonic and α-linolenic acid, which may be in an ester (e.g. ethyl ester or triglyceride) form. Other additives include herbs and plant extracts such as kelp, pectin, Spirulina, fiber, lecithin, wheat germ oil, safflower seed oil, flax seed, evening primrose, borage oil, blackcurrant, pumpkin seed oil, grape extract, grape seed extract, bark extract, pine bark extract, French maritime pine bark extract, muira puama extract, fennel seed extract, dong quai extract, chaste tree berry extract, alfalfa, saw palmetto berry extract, green tea extracts, angelica, catnip, cayenne, comfrey, garlic, ginger, ginseng, goldenseal, juniper berries, licorice, olive oil, parsley, peppermint, rosemary extract, valerian, white willow, yellow dock and yerba mate. Other additives include miscellaneous substances such as menaquinone, choline (choline bitartrate), inositol, carotenoids (beta-carotene, alpha-carotene, zeaxanthin, cryptoxanthin or lutein), para-aminobenzoic acid, betaine HCl, free omega-3 fatty acids and their esters, thiotic acid (alpha-lipoic acid), 1,2-dithiolane-3-pentanoic acid, 1,2-dithiolane-3-valeric acid, alkyl polyglycosides, polysorbate 80, sodium lauryl sulfate, flavanoids, flavanones, flavones, flavonols, isoflavones, proanthocyanidins, oligomeric proanthocyanidins, vitamin A aldehyde, a mixture of the components of vitamin A₂, the D Vitamins (D₁, D₂, D₃ and D₄) which can be treated as a mixture, ascorbyl palmitate and vitamin K₂.

The supplement or additive may be packaged for consumption in softgel, capsule, tablet or liquid form. It can be supplied in edible polysaccharide gums, for example carrageenan, locust bean gum, guar, tragacanth, cellulose and carboxymethylcellulose. Cosmetic or hygiene supplements may be provided in for example, shampoos, conditioners, creams, pastes, lotions, lipsticks, lip balms, etc.

The present invention will be further illustrated in the following examples.

### EXAMPLES

The following examples are intended to illustrate embodiments of the invention and should not be construed as limiting.

### Example 1

### Identification of Antimicrobial Agent Producing Strains

### Bacterial Strains and Growth Media

The bacterial indicator strains used are listed in Table 1. All were maintained at −80° C. in appropriate media containing 10% glycerol (w/v). P. polymyxa and all indicator strains except Butyrivibrio fibrisolvens and Fibrobacter succinogenes were propagated aerobically at 30° C. in their respective culture media as indicated in Table 1. The media used were: Tryptic soy broth (TSB) (Difco Laboratories, Sparks, M D, USA), de Man, Rogosa and Sharpe broth (MRS) (Rosell Institute, Montreal, PQ, Canada) (de Man et al. 1960) and Luria-Bertani (LB) broth. Liquid or solid (1.2% w/v agar) anaerobic L-10 medium containing glucose, maltose and soluble starch as carbon sources (each at 0.1% w/w) was used for the growth of B. fibrisolvens and F. succinogenes (Caldwell and Bryant 1966). Their growth was carried out at 39° C. in a CO₂:H2 atmosphere (95:5 v/v). Before starting the experiments, all strains were sub-cultured at least three times at 24-h intervals using 1% volume transfers.

### Isolation and Identification of Antimicrobial-Compound-Producing Bacteria

The antimicrobial agent producer P. polymyxa JB05-01-1 was isolated from a direct-fed microbial product (RE3™ Basic Environmental Systems & Technology Inc. Edmonton, AB, Canada). RE3™ was screened for bacteria producing compounds inhibiting the growth of E. coli by a deferred antagonism plating procedure as described by Tagg et al. (1976). Briefly, 100 μl of 10³-10⁴ dilutions of RE3™ in L-10 or TSB media were spread on L-10 or TSB plates and incubated overnight at 39° C. and 37° C. The plates were replicated, and then the bacterial colonies on the original plates were washed from the agar surface and the plates were surface-sterilized under Ultraviolet (UV) light at 254 nm for 20 minutes. The plates were then overlaid with 5 ml of melted LB (0.5% agar) containing 50 μl of an overnight culture of E. coli RR1 and incubated overnight at 37° C. Colonies producing clearing zones were identified and picked from the replica plates for testing for activity against E. coli O157:H7.

Gram staining, motility, catalase and oxidase tests were conducted as a preliminary step in the characterization of the selected colonies. Tentative identifications were confirmed by amplification and sequencing of 16S ribosomal RNA genes.

### DNA Extraction

The Paenibacillus strain (JB05-01-1) was grown in 3 ml of TSB at 30° C. overnight. The cells were harvested by centrifugation at 5000×g for 5 min. DNA was extracted using a Power Soil DNA Kit (MoBio Laboratories Inc., Carlsbad, Calif., USA) according to the manufacturer's instructions. The DNA concentration was measured using the PicoGreen dsDNA quantitation kit (Molecular Probes, Invitrogen, Eugene, Oreg., USA) in a Multi Detection Microplate Reader (Model SIAFRM, BioTek Instruments, Winooski, Vt., USA) using calf thymus DNA (Sigma-Aldrich, St. Louis, Mo., USA) as the standard.

Polymerase Chain Reaction Amplification of 16S rRNA Genes

The PCR amplification targeted the approximately 1500 bp of the 16S rRNA gene. The PCR reaction contained 10 ng of template DNA, 2.5 ml of 10× dilution buffer, 10 μmol of each primer and 1 U of Taq polymerase (Takara Shuzo, Japan) in a final volume of 25 ml. The primers used were the universal bacterial primers 8-27 F (5′-AGA GTT TGA TCC TGG CTC)AGA-3° (Liu et al., 1997) and 1492R (5′-TAC CTT GTT ACG ACT T-3′) (Kane et al., 1993). The amplification conditions involved denaturation at 95° C. for 1 min, followed by 25 cycles of 95° C. for 30 s, 55° C. for 30 s and 72° C. for 1.5 min. The nomenclature of the primers used was based on E. coli numbering system.

Cloning and Sequencing of 16S rRNA Genes

Amplicons from the PCR reaction were electrophoresed on 1% agarose gel and purified by excising the correct sized bands. The DNA was extracted from the gel using QIAquick PCR purification Kit (QIAGEN, Valencia, Calif.) according to the manufacturer's instructions. The purified DNA was cloned into TOPO vector (Invitrogen, Carlsbad, Calif.) and further used to transform electrocompetent E. coli (DH5-α cells) by electroporation. The cells were then plated on LB/Kanamycin (50 mg/L) agar plates and incubated overnight at 37° C. Three clones, verified for correct inserts, were grown overnight in LB/Kanamycin (100 mg/L). All clones were sequenced by the University of Calgary Core DNA Services, Calgary, AB, Canada. The 16S rRNA sequence was a consensus from three clones. The 16S rDNA sequences of the isolates were compared with DNA sequences from the National Center for Biotechnology Information (NCBI) database using the standard nucleotide-nucleotide homology search Basic Local Alignment Search Tool (BLAST) (Altschul et al., 1990).

### Results

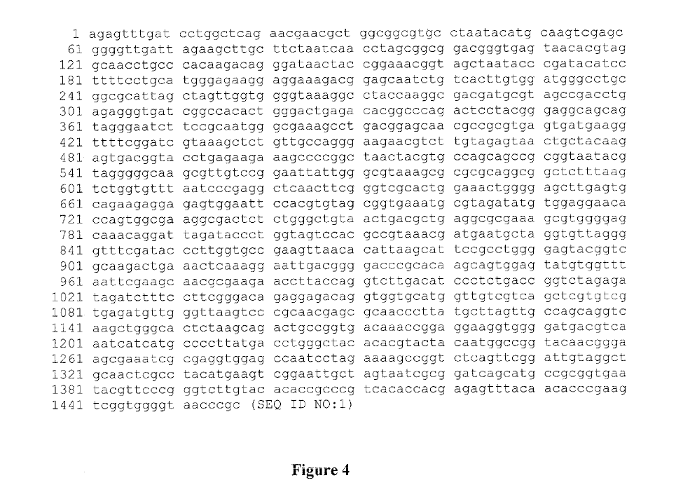

Sequencing of the 16S rDNA PCR products from the isolate showing the highest activity against E. coli O157:H7 identified an organism that shared 99% homology with Paenibacillus polymyxa. This isolate was designated as Paenibacillus polymyxa JB05-01-1. The 16S rRNA gene partial sequence of P. polymyxa JB05-01-1 was deposited with GenBank and has been assigned Accession Number GQ184435 (FIG. 4, SEQ ID NO: 1).

TABLE 1
Antimicrobial spectrum of inhibitory substance produced by Paenibacillus
polymyxa JB05-01-1 culture supernatant (CS-JB05-01-1) and polymyxin
B, used as positive control.

| | | | Inhibition* | |
|---|---|---|---|---|
| | | | CS- | Polymyxin |
| | | | JB05- | B (0.1 |
| Indicator strains | Source | Medium | 01-1 | μg ml⁻¹) |
| Bacillus circulans | LRC 9E2 | LB | − | − |
| Bacillus lecheniformis | LRC 8422 | LB | − | − |
| Butyrivibrio fibrisolvens | LRC OR85 | L-10 | + | + |
| Escherichia coli | LRC RR1 | TSB | +++ | +++ |
| Escherichia coli | LRC TB1 | TSB | ++ | +++ |
| Escherichia coli | LRC SA1650 | TSB | ++ | ++ |
| Enterococcus mundtii | LRC 8369 | LB | − | − |
| Fibrobacter succinogenes | ATCC 19169 | L-10 | + | + |
| Lactococcus lactis | ATCC 7962 | MRS | − | − |
| Listeria innocua | HPB 13 | TSB | − | − |
| Listeria ivanovii | HPB 28 | TSB | − | − |
| Pantoea agglomerans | LRC BC1 | TSB | + | + |
| Pseudomonas fluorescens | LRC R73 | TSB | ++ | ++ |
| Paenibacillus polymyxa | ATCC 43865 | LB | − | − |
| Paenibacillus polymyxa | ATCC 7070 | LB | − | − |
| Paenibacillus polymyxa | This study | LB | − | − |
| JB05-01-1 | | | | |
| Pediococcus acidilactici | ATCC 25740 | MRS | − | − |
| Pediococcus pentosaceus | ATCC 25745 | LB | − | − |
| Streptococcus bovis | ATCC 33317 | LB | − | − |

*Determined from two individual assays
− No inhibition at concentrations up of 0.1 μgml-1.
+ Diameter of the inhibition zone 10 ± 2 mm
++ Diameter of the inhibition zone 16 ± 2 mm
+++ Diameter of the inhibition zone 28 ± 2 mm
ATCC: American Type Culture Collection,
LRC: Lethbridge Research Center
HPB: Health Product Branch, (Health and Welfare Canada, Ottawa, ON, Canada)

### Example 2

### Production of the Antimicrobial Agent

One litre of LB medium was inoculated with 10 ml of a fresh, overnight culture of P. polymyxa JB05-01-1 and incubated at 30° C. with agitation at 200 rpm. The culture optical density at 600 nm was measured every two hours using a Multi-detection micro-plate reader (Bio-Teck instrument Inc., Winooski, Vt., USA), and 1 mL of culture was centrifuged (8,000 rpm, 10 min, 4° C.) to remove the cells. The supernatant was heated at 70° C. for 10 min to inactivate any protease activity, as described by Martin et al. (2003). The agar diffusion assay and micro-dilution method were used to test the heated supernatants for antimicrobial activity as described herein.

The determination of soluble protein was done using the Folin phenol reagent method as described by Lowry et al. (1951) with bovine serum albumin as standard. Polymyxin E, Polymyxin B and Nisin A were used as positive control for antimicrobial activity. Nisin A stock solutions were prepared from pure Nisin obtained from Aplin and Barrett (Beaminster, UK) in the form of Nisaplin™, which contains 2.5% (w/w) Nisin A. Polymyxin E and Polymyxin B was purchased from Sigma-Aldrich (Oakville, ON, Canada).

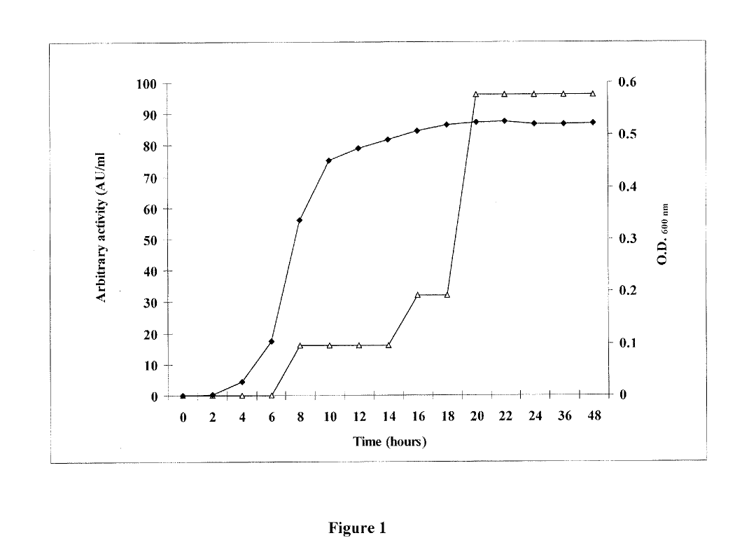

Inhibition of Escherichia coli RR1 by supernatant of a batch culture grown in Luria-Bertani broth, measured by the micro-dilution method, was maximal at 20 h and remained so through 48 h. Thus, based on agar diffusion and micro-dilution tests, the secretion of the antimicrobial agent was shown to start in the exponential phase and reach its maximum in the early stationary phase (FIG. 1). Production thus appeared to be growth-associated and activity levels remained stable through 48 h. Inhibition zone diameters at 48 h were approximately 8±1 mm and activity was 96 AU ml⁻¹.

### Example 3

### Spectrum of Activity

The qualitative antimicrobial spectrum of P. polymyxa culture supernatant was determined using the agar well diffusion method (Wolf and Gibbons 1996). Briefly, a 25-ml volume of molten tryptic soy agar (0.75% agar w/v) was cooled to 47° C. and seeded with 1% (v/v) overnight TSB culture of an indicator strain. The seeded agar was then poured into a sterile Petri plate and allowed to solidify at room temperature. Wells (7 mm) were cut in the solidified agar using a sterile metal cork borer and filled with 80 μl of supernatant. The plates were left at 5° C. for 2 h to allow diffusion of the tested aliquot and then incubated aerobically for 18 h at 30° C. Absence or presence of inhibition zones as well as their diameters were recorded.

The antimicrobial activity was also determined by the micro-dilution method described by Daba et al. (1994). Activity was expressed in arbitrary units per milliliter (AU ml⁻¹) using the formula (1000/125)^{X}(1/D), where D was the highest dilution causing inhibition of the indicator strains.

The minimum inhibitory concentration (MIC) of P. polymyxa JB05-01-1 culture supernatant and pure polymyxin E or polymyxin B against E. coli RR1 was determined using a Microtest™ polystyrene micro-plate assay (96-well, Becton Dickinson Labware, Lincoln Park, N.J.) as described by Kheadr et al., (2004).

The inhibitory spectrum of the culture supernatant is presented in Table 1. Gram-negative staining bacteria (E. coli RR1, Pantoea agglomerans BC1, Pseudomonas fluorescens R73, B. fibrisolvens OR85 and F. succinogenes S85) were inhibited while no activity was detected against Gram-positive staining bacteria. The spectrum of activity of the antimicrobial agent produced by P. polymyxa JB05-01-1 was different from that of Nisin A but similar to polymyxin E and polymyxin B used as a positive control (Table 1).

To determine the effect of P. polymyxa JB05-01-1 culture supernatant, E. coli RR1 cells were cultivated in the presence of concentrated P. polymyxa culture supernatant containing JB05-01-1 at final total activity of 0, 32, and 96 AU/ml. The OD₆₀₀ₙₘ of culture was determined every two hours using a multi-detection microplate reader (Bio-Teck instrument Inc, Winooski, Vt., USA). The corresponding final cell concentration expressed as CFU/ml was determined as described by Naghmouchi et al. (2008). The inhibitory activity (I.A.) of culture supernatant containing JB05-01-1 was calculated as a percentage, I.A.=100-100[OD₆₀₀(x)/OD₆₀₀(i)], where (x) is culture supernatant containing JB05-01-1 at the corresponded total activity and (i) is the control culture. Data was reported as means of duplicate analyses.

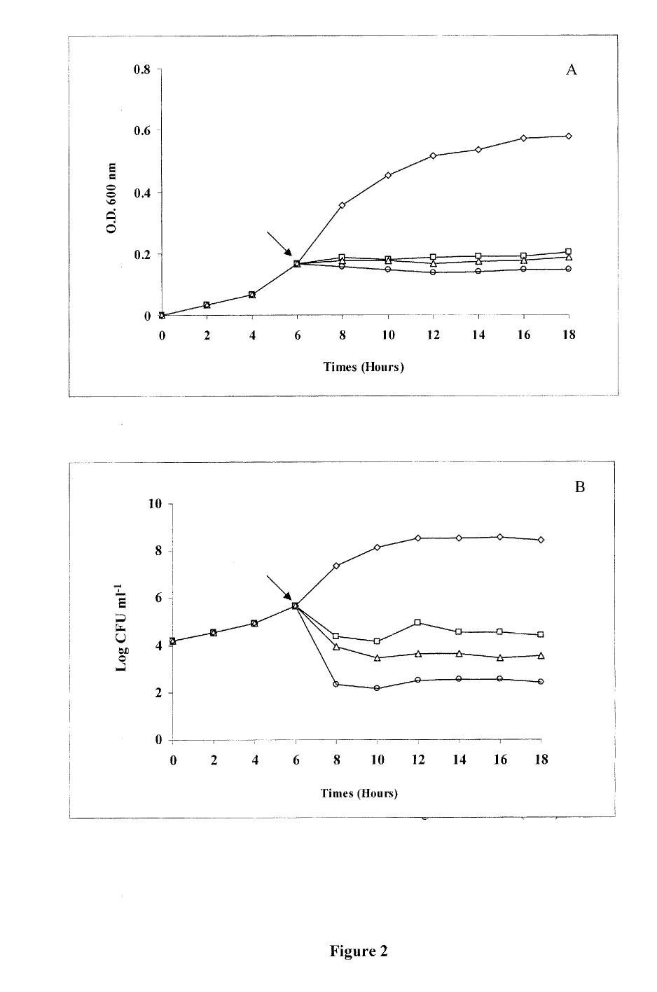

To determine the mode of action of culture supernatant containing JB05-01-1, the viability of E. coli RR1 cells was examined (FIG. 2) upon their contact with culture supernatant containing JB05-01-1. Thus, addition of culture supernatant containing JB05-01-1 at total activity of 32 or 96 AU/ml reduced the final cells concentrations of E. coli RR1 by about 2.7 and 3.4 log₁₀ CFU/ml after 2 h of exposure, respectively (FIG. 2b). The corresponding inhibitory activities were estimated to 47.1% and 51%, respectively. The OD₆₀₀ₙₘ of the cell suspension was constant through the experiments (FIG. 2a). The data obtained indicates that P. polymyxa JB05-01-1 has bactericidal action like polymyxin B, which was used as a positive control.

### Example 4

### Characterization of the Antimicrobial Agent

The sensitivities of the antimicrobial agent to proteases (all from Sigma-Aldrich, Oakville, ON) or other agents was tested by treating P. polymyxa JB05-01-1 culture supernatant with 2 mg ml⁻¹ final concentration of proteinase K (Tritirachium album), α-chymotrypsin (bovine pancreas), lipase (Sigma-Aldrich, Oakville, ON), trypsin (porcine pancreas), urea (Sigma-Aldrich, St. Louis Mo.), sodium dodecyl sulfate (SDS) (Sigma-Aldrich, St. Louis Mo.) for 1 h at 37° C. (Motta et al. 2007). Thermal stability of the antimicrobial activity was determined by holding aliquots (1000 μl) of 20 h culture supernatant at temperatures ranging from 50° C. to 90° C. for 30 min or at 100° C. for 10 min. The effect of pH was determined by adjusting the pH of P. polymyxa JB5-01-1 culture supernatant from 2 to 9 using 5 M HCl or NaOH. The activity of each sample was compared with the activity of untreated P. polymyxa JB05-01 culture supernatant at pH 6.8.

The effect of several organic solvents was evaluated by stirring 20 h culture supernatant for 2 h with 10% (v/v) acetonitrile, hexane, propanol, ethanol, toluene, acetone, butanol or methanol (all solvents were obtained from Sigma-Aldrich, St. Louis Mo.). Residual antimicrobial activities were tested using the agar diffusion assay against E. coli RR1 as described herein, with controls for effects of residual solvent.

The effect of enzymes, detergents and other compounds on the anti-E. coli activity of the P. polymyxa JB05-01-1 culture supernatant is shown in Table 2.

TABLE 2
Inhibition of Escherichia coli RR1 by Paenibacillus polymyxa
JB05-01-1 culture supernatant (CS-JB05-01-1) or polymyxin B
treated with enzymes, detergents or urea, as determined by
the agar diffusion test.

% of residual activity*

| Treatment agent | CS-JB05-01-1 | Polymyxin B (0.1 μg ml⁻¹) |
|---|---|---|
| None | 100 | 100 |
| Proteinase K | 0 | 4.5 |
| Trypsin | 83.3 | 62.5 |
| Chymotrypsin | 75 | 79.5 |
| Lipase | 62.5 | 58.3 |
| SDS | 66 | 70.1 |
| Urea | 58 | 62.5 |

*Antimicrobial activities of culture supernatant without additives are 100%. Results are means of two individual assays with an SD less than 5% about the mean.

Activity was eliminated after proteinase K treatments. Lipase, trypsin, α-chymotrypsin, sodium dodecyl sulphate (SDS) and urea reduced the antimicrobial activity by 38%, 17%, 25%, 34% and 42% respectively when compared to untreated activity.

The antimicrobial activity remained unchanged after heating at 80° C. for 30 min. Loss of activity of about 60% was observed after heating at 90° C. for 30 min. Heating to 100° C. for 10 min completely eliminated the antimicrobial activity.

Organic solvents such as chloroform, propanol, methanol, ethanol and toluene did not affect the activity of the antimicrobial peptide. Acetonitrile or hexane treatment at the same concentration (10%, v/v) reduced the antimicrobial activity by about 5% and 20%, respectively. Activity also remained stable after a two-hour incubation at pH ranging from 2 to 9.

### Example 5

### Molecular Weight Determination

P. polymyxa JB05-01-1 culture supernatant was analysed in duplicate using a NuPAGE 12% Bis-Tris gel kit (Invitrogen, Burlington, ON, Canada) as per manufacturer's instructions at 200 V (constant) for 40 min. The 2.5-200 kDa molecular weight marker kit from Invitrogen was used as a molecular weight standard. After electrophoresis, one gel was stained with Coomassie Brilliant Blue 8250 (Invitrogen). A duplicate gel was used for the plate overlay assay to estimate the molecular weight of the antimicrobial compounds as described by Bhunia et al. (1987). Briefly, a SDS-PAGE gel prewashed with sterile water was placed in a Petri dish and overlaid with 10 ml tryptic soy agar containing growing cells of E. coli RR1 at about 10⁵ CFU ml⁻¹. The agar was allowed to solidify, held at 4° C. for 60 min and then incubated for 18 h at 30° C. The formation of an inhibition zone indicated the position and size of the active antimicrobial peptide in the gel.

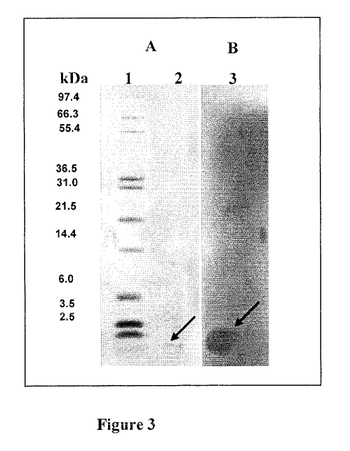

Coomassie Brilliant Blue staining of SDS-PAGE gels of P. polymyxa JB05-01-1 culture supernatant revealed no distinct protein bands. However, inhibitory activity was detected as a clearly defined zone of inhibition in the region corresponding to a molecular mass of <2.5 kDa (<2500 Da) after gels were overlaid with E. coli RR1-seeded agar (FIG. 3). No inhibitory activity was detected when the SDS gels were overlaid with Listeria innocua.

### Example 6

### Isolation and Purification of Antimicrobial Peptides

### Methods

### Strains and Culture Conditions

The bacterial strains used and their culture media are listed in Table 4. All strains were stored previously at −80° C. in media containing 20% glycerol (w/v) and were sub-cultured twice in the appropriate media prior to being used in experiments. Fresh cultures were prepared by inoculation of 10 ml of the corresponding medium with 100 μl of the frozen stock followed by incubation for 18-24 h at the corresponding temperature.

P. polymyxa JB05-01-1 was grown aerobically at 30° C. in Luria-Bertani (LB) broth (Difco Laboratories, Detroit, Mich., USA) for 16 h. Colistin-sensitive strain Escherichia coli RR1 (E. coli RR1), was obtained from the Lethbridge Research Center culture collection. E. coli RR1 was grown aerobically for 18 h at 30° C. in tryptic soy broth (TSB, Difco Laboratories, USA).

### Isolation of Antimicrobial Peptides

P. polymyxa JB05-01-1 culture was used to inoculate 500 ml of Luria-Bertani (LB) broth (1% inoculum) in a 1-liter flask and incubated at 30° C. for 20 h. Cells in the fermentation broth were separated by centrifugation (12,000 g, 20 min, 4° C.). The resulting cell-free supernatant was heated (100° C. for 10 min) and passed through a 5×50 cm column (Amersham Pharmacia Biotech) packed with 60 g Amberlite XAD-16 resin (Sigma-Aldrich, Oakville, ON, Canada) via a Gilson MiniPlus 2 Pump at a flow rate of 5 ml/min to remove hydrophobic components from the medium as described by Martin et al. (2003). The Amberlite XAD-16 column was washed with 300 ml of 30% ethanol after which the active fraction was eluted with 500 ml of 70% isopropanol (pH 2; achieved through the addition of 1 M HCl) at a flow rate of 5 ml/min. The eluted active fraction was then evaporated to 30% aqueous volume (approximately 150 ml) using a rotary evaporator (Laborota 4001 Efficient; Krackeler Scientific, Inc., Albany, N.Y., U.S.A.) at 30° C. The Amberlite XAD-16 column was washed with 500 ml of HPLC grade water for future use.

The evaporated active fraction was passed through a Waters Sep-Pak Vac 35 cc C18-10 g column (Waters, Milford, Mass., USA) via Gilson MiniPlus 2 Pump at a flow rate of 2.5 ml/min. The column was washed with 30 ml of 20% acetonitrile (CH₃CN) 0.5 M HCl and the active fraction eluted with 50 ml of 50% acetonitrile 0.5 M HCl (Selim et al. 2005). The active fraction eluted from the Sep-Pak C18 column was evaporated under similar conditions as hereinbefore described to 20 ml and further concentrated to 5 ml using Centricon YM-3 centrifugal filter unit (Millipore: Bedford, Mass., USA) at 3000 g for 120 min at 4° C. The evaporated active fraction eluted from the Sep-Pak C18 column was designated as Fraction E.

All fractions obtained at each stage of the isolation process were assessed for antibacterial activity using the micro-dilution method as described below.

### Gel Filtration Chromatography Technique

Fraction E was applied to Superdex 75 HR 16/60 gel filtration chromatography to obtain the active fraction based on molecular weight separation. The Superdex 75 HR 16/60 column was equilibrated with 100 mM sodium phosphate extraction buffer (pH 7.4) containing 150 mM NaCl. This step was carried out at 4° C. with a flow rate of 0.5 ml/min using a Fast Protein Liquid Chromatography (FPLC) system (GE Healthcare Canada Inc.). One ml of Fraction E was applied onto the column and fractions of 1 ml were collected during the elution process using an isocratic gradient. The mobile phase consisted of 30% CH₃CN/70% HPLC grade water, 0.1% trifluoroacetic acid (TFA) single buffer. Elution was monitored at wavelength detection of 218 nm during a total running time of about 600 min. Fractions exhibiting antibacterial activity, at a given retention time, were collected from different FPLC runs, pooled and lyophilized. The resulting powder was dissolved in 0.1 ml HPLC grade water and checked again for activity against E. coli RR1 and the other bacteria listed in Table 4.

### Antibacterial Activity Assay

Antibacterial activity of the fractions obtained at each stage of the isolation process was determined by the micro-dilution method (Daba et al. 1994). Total activity was expressed in arbitrary units per milliliter (AU/ml), using the formula (1000/125)×(1/D), where D was the highest dilution causing complete inhibition of E. coli RR1, the indicator strain (Naghmouchi et al. 2010).

### Protein Concentration Determination

The determination of soluble protein concentration was performed in triplicate with the Folin phenol reagent method (Lowry et al. 1951). Bovine serum albumin (BSA) was used as internal standard. Polymyxin E solution standard was obtained from Sigma-Aldrich (Oakville, ON, Canada).

### Determination of Minimum Inhibitory Concentration

Minimum inhibitory concentration (MIC) for FPLC purified peptide peak (retention time of 361.55 min) and that of polymyxin (colistin-standard) were determined by the micro-dilution assay (Naghmouchi et al. 2010).

### MALDI TOF/TOF Analysis

Two microlitres of the peak sample obtained from final FPLC purification step (retention time 361.55 min) was analysed directly by over-spotting with 1 μL α-Cyano-4-hydroxycinnamic acid (3 mg/mL containing 1.8 mg/mL ammonium citrate dissolved in 50% ACN/0.1% TFA). In addition, 5 μL of each sample was diluted with 50 μL 0.1% TFA/water C18 Ziptip desalted and eluted with matrix. The samples were air dried and then analysed on an Applied Biosystems MDS Sciex 4800 TOF/TOF Mass Analyser over mass range 600-4000 m/z collecting 500 laser shots. Ions of highest intensity were manually selected for MSMS fragmentation for 1000 laser shots and data acquired.

### Manual Nanospray Analysis

Four microliters of the peak sample obtained from final FPLC purification step (retention time 361.55 min) was placed into a silica Au/Pd coated nanoES spray tip (Proxeon, Odense. Denmark). Static manual nanospray was performed on an Applied Biosystems/MDS Sciex QStar Pulsar I fitted with a Protana/Proxeon Nanospray source. Spray was established by applying a tip voltage of 1200V and collecting a TOFMS survey scan 300-1200 m/z consisting of 50 accumulated 1 sec scans. Mass spectrometer parameters used were as follows: Declustering potential setting of 55, Focusing Potential setting of 265, Curtain gas setting of 25 and CAD gas setting of 6 with Nitrogen in the collision cell. The most intense double charged ions were selected for MSMS fragmentation. Product ion scans were acquired over the mass range 100-1500 m/z range and collision energy was manually increased to provide the best fragmentation during the 50 accumulated I second scans. Mass spectrometer parameters used for MSMS were as follows: Declustering potential setting of 50, Focusing Potential setting of 220, Curtain gas setting of 25 and CAD gas setting of 7 with Nitrogen in the collision cell.

### Results

Isolation, purification and characterization of antimicrobial substance(s) produced by P. polymyxa JB05-01

The characterization of antimicrobial substance(s) from Luria-Bertani broth cultured with P. polymyxa JB05-01-1 is summarized in Table 3.

TABLE 3
Antimicrobial substance(s) recovery at different stages of purification.
Total and partial purified substance(s) were quantified by the
micro-dilution test using E. coli RR1 as indicator strain.

| | | | | | Increase | |
|---|---|---|---|---|---|---|
| | | | | Specific | in | |
| | | Total | Total | activity² | Specific | |
| | Volume | protein | activity¹ | (AU/ | activity | Recovery |
| Step/fraction | (ml) | (mg) | (AU) | mg) | (fold) | (%) |
| Supernatant | 500 | 1750 | 48000 | 27.42 | 1 | 100 |
| Amberlate | 200 | 196 | 25600 | 130.61 | 4.8 | 53 |
| XAD-16 | | | | | | |
| Sep-Pack | 50 | 3.15 | 6400 | 2031.7 | 74.09 | 13.33 |
| C₁₈ eluate | | | | | | |
| FPLC- | 1 | 0.05 | 256 | 5120 | 189.62 | 1.06 |
| system | | | | | | |

¹Activity (AU/ml) determined by micro-titer plate assay using E. coli RR1 as indicator strain and multiplied by the volume in milliliters.
²Activity (AU/ml) divided by total protein (mg).

Extraction of the antimicrobial substance(s) was achieved using Amberlite XAD-16-adsorbent, a non-ionic macro-reticular resin that adsorbs and releases ionic species through hydrophobic and polar interactions (Zengguo et al. 2007). By applying XAD-16 resin to clarified cell-free culture supernatant, the antimicrobial substance(s) was/were selectively adsorbed, whilst other water-soluble components remained in the liquid phase. The antimicrobial substance(s) was/were eluted from XAD-16 bp 70% acid and isopropyl alcohol, and the resulting fraction was evaporated to 30% aqueous volume which retained most of the antimicrobial activity. The specific activity obtained after Amberlite XAD-16-adsorbent was approximately 130.61 AU/mg of protein.

The antimicrobial substance(s) was/were subsequently applied to Sep-Pack C18 column and the most active fraction purified with 50% acetonitrile and 0.5 M HCl. The recovery from the Sep-Pack C18 step was 13.3% with the specific activity of 2,032 AU/mg of protein.

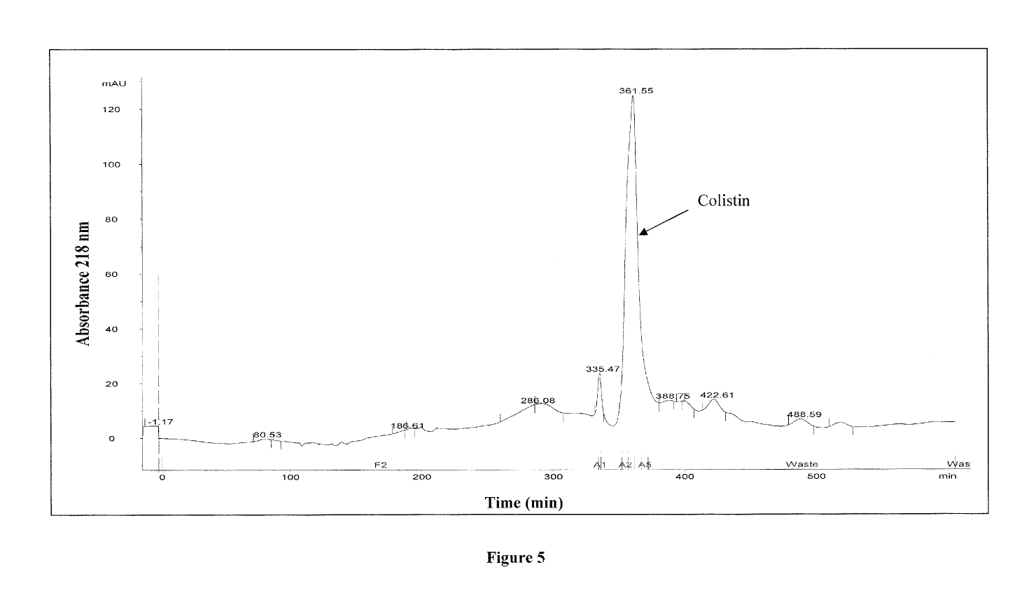

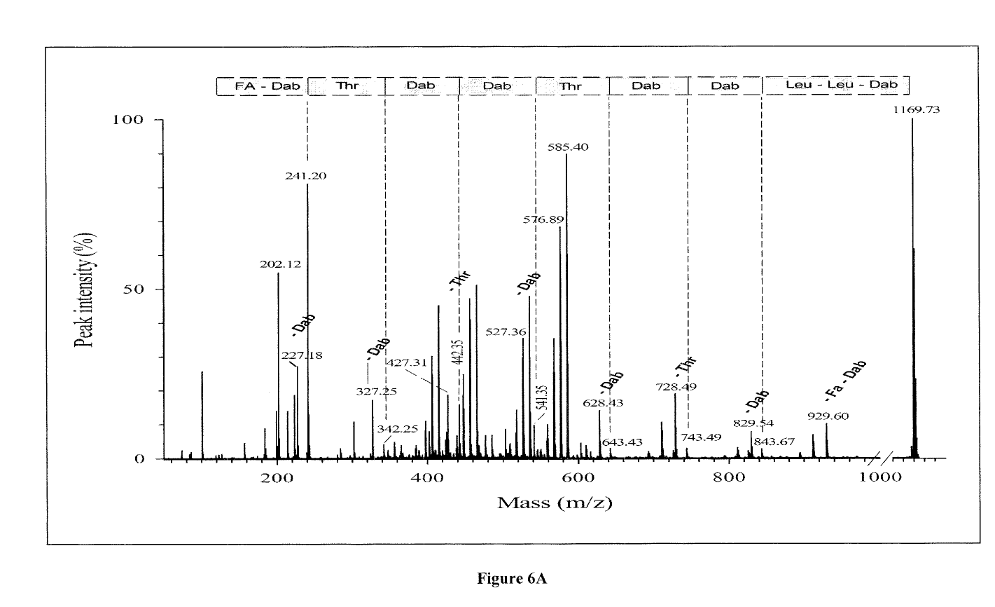

Finally, the antimicrobial fraction obtained from the Sep-Pack C18 recovery step was separated with the FPLC system connected to Hiload 16/60 Superdex 75 Prep grade size exclusion. In the FPLC profile, fractions with a peak retention time (RT) of 361.55 min were identified (FIG. 5). These fractions were active against E. coli RR1. Approximately 1.1% of the culture supernatant was recovered as antimicrobial substance(s) through the FPLC filtration process. The procedure increased the specific activity (per mg of protein) of the antimicrobial substance(s) 190-fold. Total purified antimicrobial substance(s) obtained from 500 ml of the fermented broth were approximately 0.05 mg.

The MIC of FPLC purified peptide peak (retention times of 361.55 min) against E. coli RR1 was approximately 0.13 μg/ml.

### Structure Determination

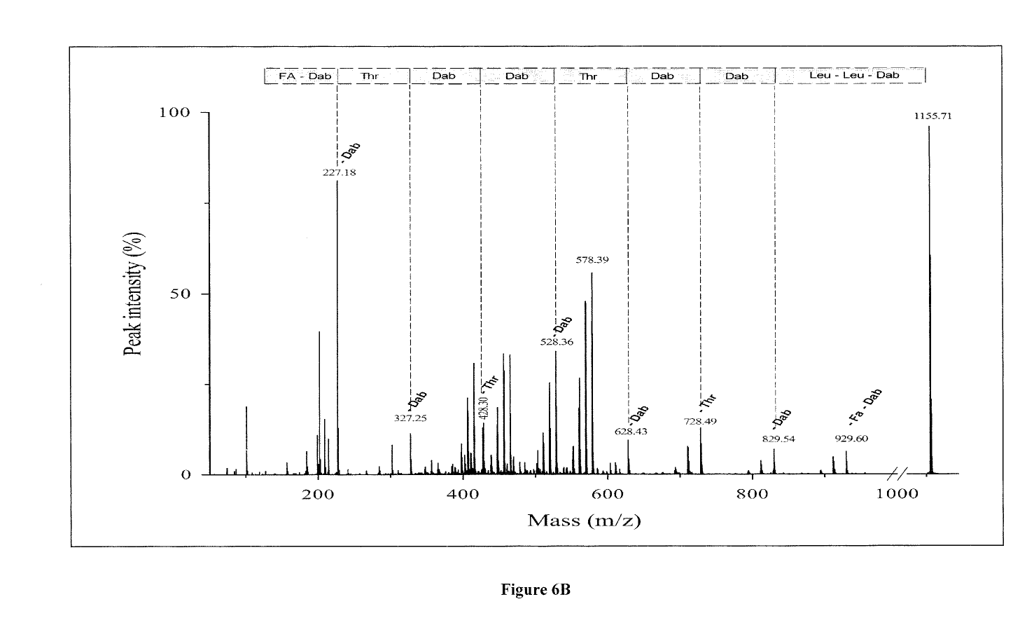

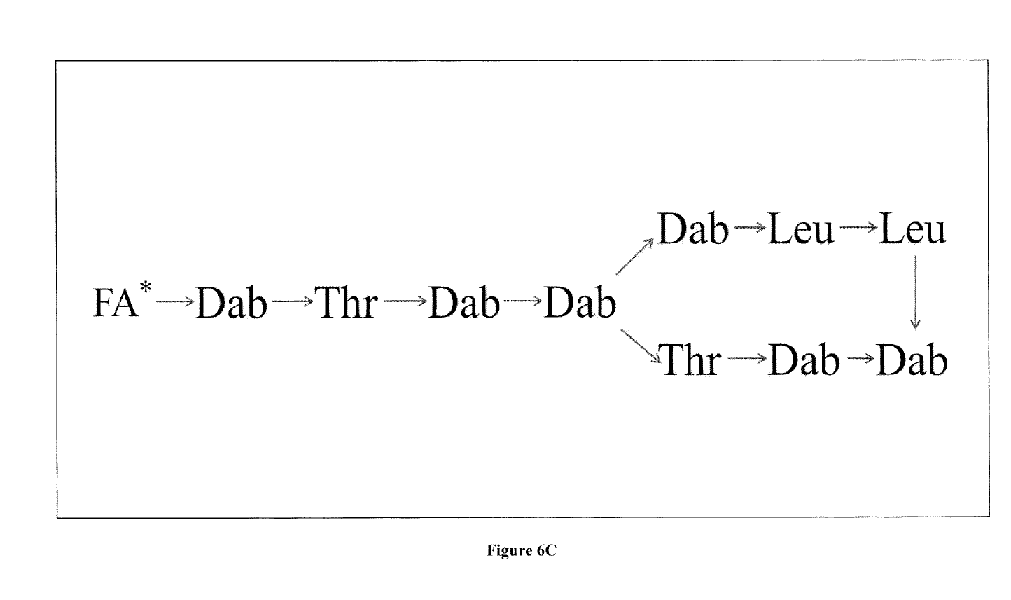

The chemical nature of the antibacterial substance(s) produced by P. polymyxa JB05-01-1 was elucidated by MALDI TOF/TOF analysis and Nanospray analysis (FIGS. 6A and 6B) as described below.

Tandem mass spectrometry MS analysis was performed for a single active peak obtained from the final FPLC purification (361.55 min) step. Data analysis revealed the presence of two compounds with the following molecular weights 1169.7 Da (FIG. 6A) and 1155.7 Da (FIG. 6B), which are equivalent to those of colistin A and colistin B. Further confirmation of this structure (colistin) was performed with MS/MS analysis on TOF and ESI. Fragmentation of peak 585.3 Da (A) in the ion trap permitted the identification in the spectrum (FIG. 6A) of the first series of product ions with m/z 829.5, 728.5, 628.4, 527.3, 427.3, 327.2, 227.2, which are formed by subsequent loss of amino acid moieties. Similar results were observed for peak 578.4 Da (FIG. 6B). The m/z ions obtained in the second series were identical to fragmentation patterns reported in the literature (Govaerts et al. 2002; De Crescenzo et al. 2007).

FIG. 6C shows the chemical structure of fatty acid colistin A and colistin B. Thus it was concluded that the active antimicrobial substance purified from P. polymyxa JB0501 are colistin A and colistin B.

### Spectrum of Activity

As shown in Table 4, purified colistin (A/B) produced by P. polymyxa JB05-01-1 had inhibitory activity against Gram-negative bacteria including E. coli O157:H7, E. coli RR1, Pseudomonas fluorescens R73 and Pseudomonas aeruginosa. The MIC values of the purified peptides were about 0.13 and 0.162 μg/ml for E. coli O157:H7 and P. fluorescens LRC R73, respectively. Similar MIC values were obtained with the colistin standard.

TABLE 4
Antimicrobial spectrum and minimal inhibitory concentration (MIC)
of purified peptide (colistins A and B) produced by Paenibacillus
polymyxa JB05-01-1

| | | | Inhibition zone* | |
|---|---|---|---|---|
| | | | attributed to | |
| | | Growth | Purified peptide | MIC |
| Indicator strains | Source | Medium | at (5 μg/ml) | (μg/ml) |
| Listeria ivanovii | HPB28 | TSB | − | 2.5-5 |
| L. monocytogenes | LSD530 | TSB | − | 5-10 |
| Escherichia coli | MC4100 | TSB | ++ | 0.13-0.26 |
| E. coli O157:H7 | ATCC | TSB | ++ | 0.13 |
| | 35150 | | | |
| E. coli | ATCC | TSB | ++ | 0.13 |
| | 25922 | | | |
| E. coli | RR1 | TSB | +++ | 0.13 |
| Pseudomonas | ATCC | TSB | +++ | 0.52 |
| aeruginosa | 19442 | | | |
| P. fluorescens | LRC R73 | TSB | +++ | 0.162 |
| Lactococcus lactic | UL719 | MRS | − | − |
| Paenibacillus | ATCC | MRS | − | − |
| polymyxa | 43865 | | | |
| Pediococcus | UL5 | MRS | − | − |
| acidilactici | | | | |
| Salmonella enterica | UL | TSB | + | 1.04-2.08 |
| Staphylococcus | Scott A3 | TSB | − | − |
| aureus | | | | |

*Determined from two individual assays
−: No inhibition at concentrations up of 5 μg ml⁻¹
+: Diameter of the inhibition zone 10 ± 2 mm
++: Diameter of the inhibition zone 16 ± 2 mm
+++: Diameter of the inhibition zone 28 ± 2 mm
ATCC: American Type Culture Collection;
UL: University Laval, Quebec, Canada
LRC: Lethbridge Research Center, Alberta, Canada;
LSD: Laboratory Services Division, Ottawa, ON, Canada; HPB: Health Product Branch, (Health and Welfare Canada, Ottawa, ON, Canada).
MRS: de Man-Rogosa and Sharpe medium
TSB: Tryptone Soya Broth medium

### REFERENCES

Altschul S F, Gish W, Miller W, Myers E W, Lipman D J. Basic local alignment search tool. J Mol Biol 1990; 215:403-410.
Bhunia A K, Johnson M C, Ray, B. Direct detection of an antibacterial peptide of Pediococcus acidilactici in sodium dodecyl sulfate-polyacrylamide gel electrophoresis. J Ind Microbiol 1987; 2:319-322.
Caldwell D R, Bryant M P. Medium without rumen fluid for non selective enumeration and isolation of rumen bacteria. Appl Microbiol 1966; 14:794-801.
Cleveland J, Montville T J, Nes I F, Chikindas M L. Bacteriocins: Safe, natural antibacterials for food preservation. Int. J. Food Microbiol, 2001; 71:11-20.
Daba H, Lacroix C, Huang J, Simard R E, Lemieux L. Simple method of purification and sequencing of a bacteriocin produced by Pediococcus acidilactici UL5. J Appl Bacteriol 1994; 77:682-688.
Davies E A, Bevis H E, Potter R, Harris J, Williams G C, Delves-Broughton J. The effect of pH on the stability of nisin solution during autoclaving. Lett Appl Microbiol 1998; 27:186-187.
de Man J C, Rogosa M, Sharpe M E. A medium for the cultivation of lactobacilli. J Appl Bacteriol 1960; 23:130-135.
DeCrescenzo H E, Phillips D R, Doran Peterson J B. Polymyxin E production by P. amylolyticus. Letters Appl Microbiol 2007; 45:491-496.
Du L, Shen B. Identification and characterization of a type II peptidyl carrier protein from the bleomycin producer Streptomyces verticillus ATCC 15003. Chem. Biol. 1999; 6:507-517.
Falagas M E, and Kasiakou S K (2005). Colistin: the revival of polymyxins for the management of multidrug-resistant gram-negative bacterial infections. Clin Infect Dis. 40:1333-1341.
Falagas M E, Kasiakou S J (2006) Toxicity of polymyxins: a systematic review of the evidence from old and recent studies. Crit. Care. 10:R27.
Fangio M F, Roura S I, Fritz R (2010) Isolation and identification of Bacillus spp. and related genera from different starchy foods. J Food Sci. 75:218-221.
Govacrts C, Orwa J. Van Schepdael A, Roets E, Hoogmartens J (2002) Characterization of polypeptide antibiotics of the polymyxin series by liquid chromatography electrospray ionization ion trap tandem mass spectrometry. J. Pept. Sci. 8:45-55.
He Z, Kisla D. Zhang L, Yuan C, Green-Church K B, Yousef A E (2007) Isolation and identification of a Paenibacillus polymyxa strain that coproduces a novel lantibiotic and polymyxin. Appl Env Microbiol. 73:168-178.
He Z, Yuan C, Zhang L, Yousef A E (2008) N-terminal acetylation in paenibacillin, a novel lantibiotic. FEBS Lett. 582:2787-2792.
Jian Li, Milnel R W, Nation R L, Turnidge J D, Smeaton T C, Coulthard K (2004) Pharmacokinetics of colistin methanesulphonate and colistin in rats following an intravenous dose of colistin methanesulphonate J Antimicrobiol Chemother. 53: 837-840.
Kane M D, Poulsen L K, Stahl D A. Monitoring the enrichment and isolation of sulfate-reducing bacteria by using oligonucleotide hybridization probes designed from environmentally derived 16S rRNA sequences. Appl Environ Microbiol 1993; 59:682-686.
Kheadr E, Bernoussi N, Lacroix C, Fliss I. Comparison of the sensitivity of commercial strains and infant isolates of bifidobacteria to antibiotics and bacteriocins, Int Dairy J 2004; 14; 273-285.
Klaenhammer T R. Genetics of bacteriocins produced by lactic bacteria. FEMS Microbiol Rev 1993; 12:39-86.
Komura S, Kurahashi K. Biosynthesis of polymyxin E by a cell-free enzyme system. J Biochem 1985; 97:1409-1417.
Lampis G, Deidda D, Maullu C, Madeddu M A, Pompei R, Delle Monachie F, Satta G (1995) Sattabacins and sattazolins: new biologically active compounds with antiviral properties extracted from a Bacillus sp. J Antibiot (Tokyo). 48:967-72.
Landman D, Georgescu C. Martin D A, Quale J (2008) Polymyxins Revisited Clinical. Microbiol. Rev. 21.449-465.
Lim L M, Ly N, Anderson D, Yang J C, Macander L, Jarkowski A 3rd, Forrest A, Bulitta J B, Tsuji B T (2010) Resurgence of colistin: a review of resistance, toxicity, pharmacodynamics, and dosing. Pharmacotherapy. 30:1279-91.
Liu W T, Marsh T L, Cheng H, Forney I J. Characterization of microbial diversity by determining terminal restriction fragment length polymorphisms of genes encoding 16S rRNA. Appl Environ Microbiol 1997; 63:4516-4522.
Lowry O H, Rosebrough N J, Farr A L, Randall R J. Protein measurement with the Folin phenol reagent. J Biol Chem 1951; 193:267.-275.
Marahiel M A, Stachelhaus T, Mootz H D. Modular peptide synthetases involved in nonribosomal peptide synthesis. Chem Rev 1997; 97:2651-2673.
Martin N I, Hu H. Moake M M, Churey J J, Whittal R, Worobo R W, Vederas JC. Isolation, structural characterization, and properties of mattacin (polymyxin M), a cyclic peptide antibiotic produced by Paenibacillus kobensis M. J. Biol. Chem. 2003; 278:13124-13132.
Motta A S, Lorenzini D M, Brandelli A. Purification and partial characterization of an antibacterial peptide produced by a novel Bacillus sp. Isolated from the amazon basin. Curr Microbiol 2007; 54:282-286.
Naghmouchi K, Drider D, Baah J, Teather R (2010) Nisin A and Polymyxin B as Synergistic Inhibitors of Gram-positive and Gram-negative Bacteria. Prob Ant Prot. 2: 98-103.
Naghmouchi K, Drider D, Fliss I. Action of divergicin M35, a class IIa bacteriocin, on Liposomes and Listeria. J Appl Microbiol. 2007; 102:1508-17.
Naghmouchi K, Drider D, Hammami R, Fliss I. (2008) Effect of Antimicrobial Peptides Divergicin M35 and Nisin A on Listeria monocytogenes LSD530 Potassium Channels. Current Microbiol 56:609-612.
Nathaniel I M, Hu H, Moake M M, Churey J J, Whittal R, Worobo R W, and Vederas J C (2003) Isolation, Structural Characterization, and Properties of Mattacin (Polymyxin M), a Cyclic Peptide Antibiotic Produced by Paenibacillus kobensis M. J Biol. Chem. 278: 13124-13132.
Nes J F, Diep D B, Havarstein L S, Bruberg M B, Eijsink V G, Holo H. Biosynthesis of bacteriocins in lactic acid bacteria. Antonie Leeuwenhock 1996; 70:113-128.
Selim S, Negrel J, Govaerts C, Gianinazzi S, van Tuinen D (2005) Isolation and Partial Characterization of Antagonistic Peptides Produced by Paenibacillus sp. Strain B2 Isolated from the Sorghum Mycorrhizosphere. Appl Environ Microbiol. 71: 501-6507.
Svetoch E A, Stern N J, Eruslanov B V, Kovalev Y N, Volodina L I, Perelygin V V, Mitsevich E V, Mitsevich I P, Pokhilenko V D, Borzenkov V N, Levchuk V P, Svetoch O E, Kudriavtseva T Y. Isolation of Bacillus circulans and Paenibacillus polymyxa strains inhibitory to Campylobacter jejuni and characterization of associated bacteriocins. J Food Prot 2005; 68:11-17.
Tagg J R, Dajani A S, Wannamaker L W. Bacteriocins of gram-positive bacteria Bacteriol Rev 1976; 40:722-56.
Wolf C E, Gibbons W R. Improved method for quantification of the bacteriocin nisin. J Appl Bacteriol 1996; 80:453-457.
Zengguo He, Kisla D, Zhang L, Yuan C, Green-Church K B, Yousef A. E. Isolation and identification of a Paenibacillus polymyxa strain that coproduces a novel lantibiotic and polymyxin. Appl Environ Microbiol 2007; 73:168-178.
Zheng G. Yan L Z, Vederas J C, Zuber P. Genes of the sbo-alb locus of Bacillus subtilis are required for production of the antilisterial bacteriocin subtilosin. J Bacteriol 1999; 181:7346-355.

### Other Embodiments

The present invention has been described with regard to one or more embodiments. However, it will be apparent to persons skilled in the art that a number of variations and modifications can be made without departing from the scope of the invention as defined in the claims. Accordingly, although various embodiments of the invention are disclosed herein, many adaptations and modifications may be made within the spirit and scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range, and of sub-ranges encompassed therein. As used herein, the terms “comprising”, “comprises”, “having” or “has” are used as an open-ended terms, substantially equivalent to the phrase “including, but not limited to”. Terms such as “the,” “a,” and “an” are to be construed as indicating either the singular or plural. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. An isolated Paenibacillus sp. bacterium comprising SEQ ID NO: 1.

2. An isolated Paenibacillus polymyxa (Strain JB05-01-1) bacterium deposited at the ATCC®) under the terms of the Budapest Treaty and designated Accession Number PTA-10436, or a strain comprising the identifying characteristics thereof.

3. The bacterium of claim 1 wherein the bacterium comprises an anti-microbial activity.

4. The bacterium of claim 3 wherein the anti-microbial activity is an anti-bacterial activity.

5. The bacterium of claim 4 wherein the anti-bacterial activity comprises inhibiting the growth of a Gram-negative staining bacterium.

6. The bacterium of claim 5 wherein the Gram-negative staining bacterium is selected from the group consisting of one or more of Escherichia sp., Pantoea sp., Pseudomonas sp., Butyrivibrio sp., Fibrobacter sp., Salmonella sp., Shigella sp., Helicobacter sp., and Campylobacter sp.

7. The bacterium of claim 5 wherein the Gram-negative staining bacterium is selected from the group consisting of one or more of Escherichia coli RR1, Escherichia coli TB1, and Escherichia coli O157:H7, Pantoea agglomerans BC1, Pseudomonas fluorescens R73, Butyrivibrio fibrisolvens OR85, Fibrobacter succinogenes and Pseudomonas aeruginosa.

8. The bacterium of claim 1 wherein the bacterium does not inhibit the growth of a Gram-positive staining bacterium.

9. A cell culture comprising the bacterium of claim 1.

10. A cell culture supernatant derived from growing the bacterium of claim 1 in a cell culture medium.

11. The cell culture supernatant of claim 10 wherein the supernatant comprises an anti-microbial activity.

12. The cell culture supernatant of claim 11 wherein the anti-microbial activity is an anti-bacterial activity.

13. An anti-microbial agent isolated from the bacterium of claim 1.

14. The anti-microbial agent of claim 13 wherein the anti-microbial agent comprises a peptide.

15. The anti-microbial agent of claim 14 wherein the peptide comprises a lipopeptide.

16. The anti-microbial agent of claim 13 wherein the anti-microbial agent comprises a molecular weight between about 1000 daltons to about 2500 daltons.

17. The anti-microbial agent of claim 13 wherein the anti-microbial agent comprises a polymyxin.

18. The anti-microbial agent of claim 13, wherein the anti-microbial agent comprises at least one of colistin A and colistin B.

19. An anti-microbial composition comprising:
an isolated Paenibacillus sp. bacterium comprising SEQ ID NO: 1;
an isolated. Paenibacillus polymyxa (Strain JB05-01-1) bacterium deposited at the ATCC®) under the terms of the Budapest Treaty and designated Accession Number PTA-10436, or a strain comprising the identifying characteristics thereof;
a cell culture comprising the bacterium;
a cell culture supernatant derived from growing the bacterium in a cell culture medium; or
an anti-microbial agent isolated from the bacterium, the cell culture or the cell culture supernatant.

20. A pharmaceutical, veterinary, cosmetic or hygiene composition comprising the anti-microbial composition of claim 19 and a suitable carrier.

21. A food or feed additive comprising the anti-microbial composition of claim 19.

22. A packaging material comprising the anti-microbial composition of claim 19.

23. A kit comprising the anti-microbial composition of claim 19 together with instructions for use in inhibiting growth of a micro-organism.

24. A method of inhibiting the growth of a microorganism in a subject or substance in need thereof, the method comprising administering or applying an effective amount of the anti-microbial composition of claim 19 to the subject or substance.

25. The method of claim 24 wherein the microorganism is a bacterium.

26. The method of claim 25 wherein the bacterium is a Gram-negative staining bacterium.

27. The method of claim 26 wherein the Gram-negative staining bacterium is selected from the group consisting of one or more of Escherichia sp., Pantoea sp., Pseudomonas sp., Salmonella sp., Shigella sp., Helicobacter sp., Campylobacter sp., Butyrivibrio sp., and Fibrobacter sp.

28. The method of claim 26 wherein the Gram-negative staining bacterium is selected from the group consisting of one or more of Escherichia coli RR1, Escherichia coli TB1, and Escherichia coli O157:H7, Pantoea agglomerans BC1, Pseudomonas fluorescens R73, Butyrivibrio fibrisolvens OR85, Fibrobacter succinogenes and Pseudomonas aeruginosa.

29. The method of claim 24 wherein the microorganism is a food-borne pathogenic micro-organism or a food-spoilage micro-organism.

30. The method of claim 24 wherein the subject is a human or an agricultural animal.

31. The method of claim 24 wherein the substance is selected from the group consisting of a cosmetic product, a hygiene product, a feed product, a food product and packaging material thereof.

32. The method of claim 31 wherein the food product is a dairy or mea product.

33. An isolated nucleic acid molecule comprising SEQ ID NO: 1.


SEQUENCE LISTING (1 sequence)

SEQ ID NO: 1 - DNA, 1457 bp, Paenibacillus polymyxa isolate JB05-01-1
agagtttgatcctggctcagaacgaacgctggcggcgtgcctaatacatgcaagtcgagcggggttgattagaagcttgcttctaatcaacctagcggcggacgggtgagtaacacgtaggcaacctgcccacaagacagggataactaccggaaacggtagctaatacccgatacatccttttcctgcatgggagaaggaggaaagacggagcaatctgtcacttgtggatgggcctgcggcgcattagctagttggtggggtaaaggcctaccaaggcgacgatgcgtagccgacctgagagggtgatcggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtcttgtagagtaactgctacaagagtgacggtacctgagaagaaagccccggctaactacgtgccagcagccgcggtaatacgtagggggcaagcgttgtccggaattattgggcgtaaagcgcgcgcaggcggctctttaagtctggtgtttaatcccgaggctcaacttcgggtcgcactggaaactggggagcttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagatctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgc